SEQUENCE LISTING (19 sequences)

SEQ ID NO: 1 - DNA, 2231 bp, Antirrhinum majus
aaattacattgcttcctttgtccccccttccaccaccaatatatacaacttcctcagctagttgtttattatcaatcaaataaaattatttcccaatgttcaaaaatcctaatatccgctatcacaaactatcttccaaatccaatgacaacgatcaagaatcctcccatcgttgtaagcacattctattatttataataaccttattcctacttatagttggcctgtacatcgccaactctctcgcctatgcccggtttgcctcgacctcaaccggccctatcgccgcccctgatgtcaccaaatgtggtcagccagacttgccacctggcacagccccaataaactgttgtcccccaatccccgctaaaatcatcgatttcgagctaccacctccctccactaccatgagggttcgccgtgcggctcatttagttgatgatgcatacattgccaaattcaagaaagccgttgagcttatgcgagctctacctgaggatgaccctcgtagcttcaagcaacaagctaacgtccattgcgcttactgcgcgggggcgtataatcaagccggtttcacaaacctaaagctccaaatccaccgatcttggctttttttcccgttccatagatattatatctacttttttgaaagaatattgggaaaactaatcaatgatacaacttttgctctcccattttggaactatgattcacctggtggaatgacaatcccatcaatgtttattgatactaattcttcgctgtacgatagtttacgggacagtaatcatcagccaccaaccatcgtagacttgaactacgccttttctgattccgacaataccactactcctgaagagcaaatgattataaaccttaaaattgtgtacagacaaatggtgtcgagcgctaagactccacagcttttcttcggccgcccataccgacgtggggaccaagagtttcccggggtggggtcgattgagttagtccctcatggcatgatacatttatggaccggttctgagaacacgccctatggcgagaacatgggggctttctactcaacggctagagacccgatattttttgctcatcattcgaacgtcgatagaatgtggtccatatggaagaccctaggagggccgcggaggacggacttaacagatccagattttcttgatgcgtctttcgttttttatgacgaaaacgcagagatggttcgggtcaaggttcgggattgcttagatgaaaagaaactagggtacgtttatcaagatgtggagattccgtggctcaacactcgtccaacaccaaaagtttctccgtctctacttaagaaatttcatagaacaaacactgccaatccgagacaagtttttcctgcgatacttgacaggtattgaattttcgtcttatctaaaagatcaagctcttagagagaattgacatttattaatttatttacgttctttcatatgccccctcatattcaggccgtaaaccttttcgttacggcttaacgtgaccttttttgatgagtgcctggccgtggaattcaaacacatgctcttatttcacttgagctctgatactatattgaactgtcgtctcacctacaaactcaaacttgtagagatgattgagatttgttacttacttatgtttttcaacaacagagtcttaaaagttatcgtgacgaggccgaagaaaactagaagtaggaaagaaaaggacgagttagaagagattttagtgattgaagggattgaactggaaagagaccacgggcacgtaaaattcgacgtttatattaatgctgacgaagatgaccttgcggtgatttcgccggagaatgctgagttcgccgggagtttcgtgagtctgtggcacaaacctataaaggggaagaggacaaagacgcagttattaacattgtcgatttgtgatattttggaggatttggatgctgacgaagatgattatgtgttggtcactttggttccgagaaacgccggagatgcgatcaagattcataatgtcaagattgagcttgatggctaataaattctattgatttcttctcaacctacagttgatcatttaccgattgattattccaataaaagtatctcatgtaccaatatcgatcgtattaatcgtaatactttcagatttttatttatttaaaagcagttgtataaatggtgaaataaggattactttttgag

SEQ ID NO: 2 - DNA, 1374 bp, Antirrhinum majus
atgggagaagaatacaagaaaacacacacaatagtctttcacacttcagaagaacacctcaactcttcaatagcccttgcaaagttcataaccaaacaccactcttcaatctccatcactatcatcagcactgcccccgccgaatcttctgaagtggccaaaattattaataatccgtcaataacttaccgcggcctcaccgcggtagcgctccctgaaaatctcaccagcaacattaataaaaaccccgtcgaacttttcttcgaaatccctcgtctacaaaacgccaaccttggagaggctttactagatatttcgcgaaaatccgatatcaaagcattaatcatcgatttcttctgcaatgcggcatttgaagtatccaccagcatgaacatacccacttacttcgacgtcagtggcggcgcttttctcctctgcacgtttctccaccacccgacactacaccaaactgttcgtggagacattgcggatttgaacgattctgttgagatgcccgggttcccattaattcactcctctgatttaccaatgagtttgttttatcgtaagagtaatgtttacaaacactttctagacacttccttaaacatgcgcaaatcgagtgggatactcgtgaacacgtttgttgcgctcgagtttcgagctaaggaagctttgtccaacggtttgtacggtccaactccgcctgtttatttactttcacatacaattgccgaaccccacgacactaaagtgttggtaaaccaacacgactgcctatcatggcttgatttgcagcctagtaaaagcgtgattttcctttgtttcggaagaagaggagcgttctcagcacaacagttgaaagaaattgccatagggttggagaagagtggatgtcgatttctttggttggcccgcatttcaccggagatggacttaaatgcgcttctgccggagggttttttatcgagaactaaaggagtagggtttgtgacaaacacatgggtgccgcagaaagaggtgttgagtcatgatgcagcgggggggtttgtgactcattgtgggtggaattctgttcttgaagcgctgtcgttcggtgtcccgatgattggttggccgttgtacgcagagcagaggatcaatagggtgttcatggtggaggaaataaaggtggcactgccattggatgaggaagatggatttgtgacggcgatggagttggagaagcgcgtcagggagttgatggagtcggtaaaggggaaagaagtgaagcgccgtgtggcggaattgaaaatctctacaaaggcagccgtgagtaaaggtggatcgtccttggttgctttggagaagttcatcaactcggtcactcgttaa

SEQ ID NO: 3 - DNA, 576 bp, Nicotiana tabacum
agtgctcttccttttctcgccgctaaatggaaaagcaaaagctcagaggagttggctaattcactcgactttttcagggatatcgtcacaggtccctttgagaaattcacccgagtgactatgatcttgcctttgacgggtaagcaatactcagagaaggtggcagaaaattgtgttgcccattggaaagcaataggaacctacaccgatgcagagagtcaggccattgaaaagctcctcaacattttccagaatgaaaccttcccgccgggtgcctccattctttttactcaatcacctgttggggcattgacgattagcttcattaaagatgattcaattactggcactggaaatgctgttatagagaacaaacaattgtctgaagcagtgctggaatccataattggcaaacatggagtttcccctgcagcaaagtgtagtatcgccgaaagagtgtcaggactattcaaaaagagctatgccgacgcgtcagtttttgaaaaaccaggaattgagaaatcctccgatccagtgattgaggagaaacctaccattccagaaattggagtctag

SEQ ID NO: 4 - DNA, 576 bp, Nicotiana tabacum
ctagactccaatttctggaatggtaggtttctcctcaatcactggatcggaggatttctcaattcctggtttttcaaaaactgacgcgtcggcatagctctttttgaatagtcctgacactctttcggcgatactacactttgctgcaggggaaactccatgtttgccaattatggattccagcactgcttcagacaattgtttgttctctataacagcatttccagtgccagtaattgaatcatctttaatgaagctaatcgtcaatgccccaacaggtgattgagtaaaaagaatggaggcacccggcgggaaggtttcattctggaaaatgttgaggagcttttcaatggcctgactctctgcatcggtgtaggttcctattgctttccaatgggcaacacaattttctgccaccttctctgagtattgcttacccgtcaaaggcaagatcatagtcactcgggtgaatttctcaaagggacctgtgacgatatccctgaaaaagtcgagtgaattagccaactcctctgagcttttgcttttccatttagcggcgagaaaaggaagagcact

SEQ ID NO: 5 - DNA, 755 bp, Lactuca sativa
gcaggtacaactagacaccgaacttttcaaattttgaaatttgtccctgatactgtatcacttagtctttgatagaacgaagaactccatgaacaaggtcttcattacatgtgatcaatttcttcttgattgtggaatatggaagcattcctttctctctgcaactatcaattgctcctttgaacatctcctccaaatcatacttaaacttgaaccccatatcagttaatttctttgatgaaaaagaaactacgggtagctcagcatcaatccctggaaactttgttggaacttggtattcaggccatttctcattgatcattcttgccaattgatgaatggtggcttcatgggaagaacaaatgtatctcccttcggcttttgggttctcatatagatatatatgggactcacaaagatcatccaaatgcacatattgaccttgttttatgatcgaatagtgtgatttctcaccattgattaaagaaagtgcggtaataaggcttggagggaatgagggagtgatgaatggaccaacgactaacgttggtatgatactaatgaaatcgatattgttttcctttgttgcttcaaatgctgctttttctgccaatgtttttgatacgaaatacatccatgcagtcattttcttggagtagatgaagtccaaatcgctccaatgagactcgtcatagaccggaagttgatcatttccgtgcacgttcactgtccccgcagaggatgtaaacaccaat

SEQ ID NO: 6 - DNA, 542 bp, Lactuca sativa
agtcgttggtccattcatcactccctcattccctccaagccttattaccgcactttctttaatcaatggtgagaaatcacactattcgatcataaaacaaggtcaatatgtgcatttggatgatctttgtgagtcccatatatatctatatgagaacccaaaagccgaagggagatacatttgttcttcccatgaagccaccattcatcaattggcaagaatgatcaatgagaaatggcctgaataccaagttccaacaaagtttccagggattgatgctgagctacccgtagtttctttttcatcaaagaaattaactgatatggggttcaagtttaagtatgatttggaggagatgttcaaaggagcaattgatagttgcagagagaaaggaatgcttccatattccacaatcaagaagaaattgatcacatgtaatgaagaccttgttcatggagttcttcgttctatcaaagactaagtgatacagtatcagggacaaatttcaaaatttgaaaagttcggtgtctagttgtacctgc

SEQ ID NO: 7 - DNA, 992 bp, Solanum tuberosum
gatccatagagctcatgaacagtacatctatgtcttcattgggagtgaggaaaggttcatggactgatgaagaagattttcttttaagaaaatgtattgataagtatggtgaaggaaaatggcatcttgttcctgctagagctggtaattaaactaactaccgtgctattttatctgtctgtctcattttatgtgacattctttgtaaaatgtatgtacgtgcaggtctgaatagatgtcggaaaagttgtagactgaggtggctgaattatctaaggccacatatcaagagaggtgactttgctccggatgaagtggatctcattttgaggcttcataagctcttaggcaacaggcatgctagtttatgttttgacaaatttgattaatataatatatatgtgtgactatttcatctaaacgttacgttattatatgtagatggtcacttattgctggtagacttccaggaaggacagcaaacgatgtgaaaaactattggaacacaaaccttctaaggagtaaggtaaatattactactaaatttgttcctcatgaaaagattaacaataagtgtggagaaattactaagaatgaaataataaaacctcaaccacgaaagtatttctcaagcacaaagaagaatattacaaacaatattgtaattgtggacaaggaggaacattgcaaggaaataataagtgagaagcaaactccagatgcattgatggaaaacgtagatcaatggtggacaaatttactggaaaattgcaatgacgatgttgaagaagaagaagaagaagctgtaactaattatgaaaaaacacttacaagtttgttaaatggtgaaggtaactccatgcaacaaggacaaataagtcatgaaagttggggtgacttttctcttaatttaccacccatgcaactaggagaaaatgatgatttttctgctgaaattgacttatggaatctacttgattaacccgggataa

SEQ ID NO: 8 - DNA, 32 bp, Artificial Sequence
  Description of Artificial Sequence Synthetic primer
ggatccaaattacattgcttcctttgtcccac

SEQ ID NO: 9 - DNA, 30 bp, Artificial Sequence
  Description of Artificial Sequence Synthetic primer
aagcttctcaaaaagtaatccttatttcac

SEQ ID NO: 10 - DNA, 31 bp, Artificial Sequence
  Description of Artificial Sequence Synthetic primer
ggatccatgggagaagaatacaagaaaacac

SEQ ID NO: 11 - DNA, 28 bp, Artificial Sequence
  Description of Artificial Sequence Synthetic primer
actagtttaacgagtgaccgagttgatg

SEQ ID NO: 12 - DNA, 32 bp, Artificial Sequence
  Description of Artificial Sequence Synthetic primer
agatctctagactccaatttctggaatggtag

SEQ ID NO: 13 - DNA, 29 bp, Artificial Sequence
  Description of Artificial Sequence Synthetic primer
ctcgagagtgctcttccttttctcgccgc

SEQ ID NO: 14 - DNA, 31 bp, Artificial Sequence
  Description of Artificial Sequence Synthetic primer
ctcgaggagtccattaccattgagaattacg

SEQ ID NO: 15 - DNA, 31 bp, Artificial Sequence; identical to SEQ ID NO: 14
  Description of Artificial Sequence Synthetic primer

SEQ ID NO: 16 - DNA, 26 bp, Artificial Sequence
  Description of Artificial Sequence Synthetic primer
ggatccgcaggtacaactagacaccg

SEQ ID NO: 17 - DNA, 28 bp, Artificial Sequence
  Description of Artificial Sequence Synthetic primer
ccatggattggtgtttacatcctctgcg

SEQ ID NO: 18 - DNA, 26 bp, Artificial Sequence
  Description of Artificial Sequence Synthetic primer
actagtgcaggtacaactagacaccg

SEQ ID NO: 19 - DNA, 26 bp, Artificial Sequence
  Description of Artificial Sequence Synthetic primer
ccatggagtcgttggtccattcatca

(19) United States
(12) Patent Application Publication
(10) Pub. No.: US 2013/0305408 A1
(43) Pub. Date: Nov. 14, 2013

(54) AUREUSIDIN-PRODUCING TRANSGENIC PLANTS

(71) Applicant: J.R. SIMPLOT COMPANY, Boise, ID (US)

(72) Inventors: Caius M. ROMMENS, Boise, ID (US); Roshani SHAKYA, Boise, ID (US); Jingsong YE, Boise, ID (US)

(21) Appl. No.: 13/829,691

(22) Filed: Mar. 14, 2013

Related U.S. Application Data
(60) Provisional application No. 61/646,020, filed on May 11, 2012.

(51) Int. Cl. C12N 15/82

(52) U.S. Cl. CPC C12N 15/825
USPC 800/278; 800/298; 435/320.1

(57) ABSTRACT

Aurone, including aureusidin-6-O-glucoside, are known to have antioxidant properties. The compounds are produced in the flowers snapdragon (e.g., Antirrhinum majus) and have been suggested for potential medicinal use. The present methods use recombinant and genetic methods to produce aurone in plants and plant products. In particular, the present methods have resulted in the production of aureusidin-6-O-glucoside in the leaves of various plants.

### FIELD OF THE INVENTION

The present inventive technology concerns genetic modifying the aurone biosynthetic pathway of crop plants.

### BACKGROUND

Aurones are flavonoids with a 5-membered C-ring that provide a bright yellow color to the petals of some varieties of snapdragon (Antirrhinum), morning glory (Ipomoea), Dahlia and Coreopsis (Saito, 1990; Iwashina, 2000). An analysis of flower color variation in natural populations of snapdragon suggests that aurones play a role in fertilization and seed set by attracting pollinators (Whibley et al., 2006). Indeed, the patterning of aurone pigmentation is thought to provide a nectar guide for pollinating bumblebees (Harborne and Smith 1978, Lunau et al. 1996). In addition to this role in pigmentation, aurones have been described as phytoalexins that are used by the plant as defense agents against various pathogens; they were found to exhibit antiviral, antiparasitic, and antifungal activities (Boumendjel, 2003).

Previously, a two-step mechanism involving the oxidation of isoliquiritigenin by a hydrogen peroxide (H₂O₂)-dependent peroxidase (PRX), followed by dehydration of the intermediate compound to form aurone 4′,6-dihydroxyaurone was proposed for aurone biosynthesis in soybean (Soja hispida) seedlings (Wong E, 1966; Rathmell and Bendall, 1972). In snapdragon, the aurone aureusidin-6-O-glucoside (AOG) is produced by glucosylation of 2′,4′,6′,4-tetrahydroxychalcone (naringenin chalcone), which facilitates transport of this compound from cytoplasm to vacuole (Ono et al., 2006), followed by cyclization of the carbon bridge. The proteins involved in these reactions are chalcone 4′-O-glucosyltransferase (Am4′CGT) and the copper-containing glycoprotein aureusidin synthase (AmAs1) (Nakayama et al., 2000), respectively. Ectopic expression of the Am4′CGT and AmAs1 genes in the related plant species Torenia hybrid resulted in the petal-specific formation of trace amounts of AOG (Ono et al., 2006). The simultaneous silencing of anthocyanin biosynthesis increased AOG formation to levels that are visible as a yellow hue (Ono et al., 2006).

Although commercial interests in aurones are currently limited to how these compounds affect flower color, their antioxidant activities suggest future medicinal applications as well (Milovanovic et al., 2002; Boumendjel, 2003; Detsi et al., 2009). Indeed, the 3′,4′,6,7 tetrahydroxyaurone from Coreopsis is more effective at scavenging free radicals than vitamin C, vitamin E, and resveratrol (Venkateswarlu et al., 2004). The ability to produce aurones synthetically (Wong, 1966; Rathmell and Bendall, 1972) opens up the way to use them as dietary supplements. However, there is a preference to use naturally produced compounds, because supplement use has been linked to increased mortality (Bjelakovic and Gluud, 2007).

In the present invention, the aurone biosynthetic pathway was transferred from ornamental flowers to the leaves of crop plants. The results disclosed herein demonstrate that this modification altered the color of leaves and also enhanced their antioxidant activity.

### SUMMARY OF THE INVENTION

One aspect of the present invention concerns modifying a plant, such as a crop plant, to express one or more antioxidants that are not normally expressed or produced in the plant, or are expressed or produced at low levels in the plant. In one embodiment, the modification encompasses expression at least one of a chalcone 4′-O-glucosyltransferase gene (Am4′CGT) and an aureusidin synthase (AmAs1) gene in plants that do not normally express either gene.

Another aspect of the present invention is a plant comprising in its genome at least one of a chalcone 4′-O-glucosyltransferase gene (Am4′CGT) and an aureusidin synthase (AmAs1) gene, wherein the plant genome does not naturally comprise the Am4′CGT or AmAs1 gene.

In another embodiment, the plant genome does comprise at least one of the Am4′CGT or AmAs1 gene but either does not express these genes or expresses these genes at low levels. The present inventive methods disclosed herein encompass operably linking one or both of the Am4′CGT or AmAs1 genes to a promoter functional in plants and introducing the resultant construct into the plant, wherein the promoter expresses the Am4′CGT and/or AmAs1 gene in the plant to which it is operably linked and changes leaf color and antioxidant production, compared to an untransformed plant.

In one embodiment, the expression of one or both of the Am4′CGT or AmAs1 genes in the transformed plant is transient. In another embodiment, the expression of one or both of the Am4′CGT or AmAs1 genes in the transformed plant is constitutive. In another embodiment, the expression of one or both of the Am4′CGT or AmAs1 genes in the transformed plant is inducible.

One aspect of the present invention comprises transforming a plant with a construct that comprises (i) a promoter functional in plant tissue, operably linked to a nucleotide sequence encoding either or both of (ii) an Am4′CGT protein, or (iii) an AmAs1 protein, wherein the color of the transformed plant's leaves are different than that of an untransformed plant of the same species, and/or the leaves of the transformed plant comprise higher super oxide dismutase (SOD) inhibiting and oxygen radical absorbance capacity (ORAC) activities than control leaves. In one embodiment, the promoter is functional in plant leaves. In one embodiment, the promoter is a leaf-specific promoter.

In one embodiment the construct comprises one expression cassette, which comprises a promoter functional in plant tissue, operably linked to a nucleotide sequence encoding an Am4′CGT protein.

In another embodiment the construct comprises one expression cassette, which comprises a promoter functional in plant tissue, operably linked to a nucleotide sequence encoding an AmAs1 protein.

In another embodiment the construct comprises one expression cassette, which comprises a promoter functional in plant tissue, operably linked to a nucleotide sequence encoding an AmAs1 protein, and a second expression cassette, which comprises a promoter functional in plant tissue, operably linked to a nucleotide sequence encoding an Am4′CGT protein.

Another aspect of the present invention comprises transforming a plant with two or multiple constructs, wherein one construct comprises a promoter functional in plant tissue, operably linked to a nucleotide sequence encoding an Am4′CGT protein, and a second construct that comprises a promoter functional in plant tissue, operably linked to a nucleotide sequence encoding an AmAs1 protein.

One aspect of the present invention is a transformed plant whose leaves are different than that of an untransformed plant of the same species, and/or the leaves of the transformed plant comprise higher super oxide dismutase (SOD) inhibiting and oxygen radical absorbance capacity (ORAC) activities than control leaves.

In one embodiment, the plant that is transformed with one or more constructs according to the present invention is a leaf vegetable. In one embodiment, the leaf vegetable is selected from the group consisting of China Jute, Climbing wattle, Paracress, Common Marshmallow, Purple amaranth, Common amaranth, Prickly amaranth, Amaranth, Slender amaranth, Celery, Garden orache, Bank cress, Chik-nam, Kra don, Indian spinach, Chard, Sea Beet, Common Borage, Abyssinian Cabbage, Indian mustard, Rutabaga, Rape Kale, Black Mustard, Wild Cabbage, Kale, Kai-Ian, Cauliflower, Cabbage, Brussels Sprouts, Broccoli, Turnip, Wild turnip, Bok Choi, Chinese Savoy, Mizuna, Napa Cabbage, Rapini, Rampion, Harebell, Caper, Wild Coxcomb, Asian pennywort, Gotukola, Lamb's Quarters, American Wormseed, Southern Huauzontle, Good King Henry, Tree Spinach, Oak-Leaved Goosefoot, Huauzontle, Quinoa, Red Goosefoot, Garland chrysanthemum, Endive, Curly endive, Broad-leaved endive, Chicory, Radicchio, Cabbage thistle, Miner's lettuce, Siberian spring beauty, Ivy Gourd, Taro, Jew's mallow, Cilantro, Coriander, Sea kale, Redflower ragleaf, Phak tiu som or Phak tiu daeng, Samphire, Chipilín, Mitsuba, Caigua, Cardoon, Vegetable fern, Arugula, Lesser jack, Bhandhanya, Culantro, Fennel, Scarlina, Gallant Soldier, Ground Ivy, Lotus sweetjuice, Melindjo, Okinawan Spinach, Sea purslane, Shortpod mustard, Sea sandwort, Fishwort, John's Cabbage, Shawnee Salad, Spotted Cat's-ear, Catsear, Golden samphire, Elecampane, Water Spinach, Sweet Potato, Lablab, Indian Lettuce, Lettuce, Celtuce, Prickly Lettuce, Bottle Gourd, Dragon's head, White deadnettle, Henbit deadnettle, Red deadnettle, Nipplewort, Bush Banana, Hawkbit, Field pepperweed, Dittander, Maca, Garden cress, Virginia pepperweed, Decne, Phak kratin, Lovage, Genjer, Rice paddy herb, Ngó om, Gooseneck Loosestrife, Cheeseweed, Mallow, Musk Mallow, Cassaya, Kogomi, duo rui gao he cai, Japanese mint, Habek mint, Sea bluebell, Ice plant, Seep monkey flower, Mauka, Drumstick tree, South-west African moring a, Ethiopian moring a, Wall lettuce, Ujuju, Parrot feather, Cicely, Watercress, Phak chet, Fragrant Water Lily, Water Snowflake, Yellow floating heart, Sweet Basil, That basil, Lemon basil, Water Celery, Common evening primrose, Hooker's Evening-primrose, Sensitive fern, Pheka, Rice, Cinnamon fern, Interrupted fern, Common wood sorrel, Creeping woodsorrel, Iron Cross, Redwood sorrel, Common yellow woodsorrel, Oca, Mountain sorrel, Money tree, Petai, Blue Palo Verde, Parsnip, Golden lace, Empress tree, Burra Gookeroo, Clearweed, Barbados Gooseberry, Perilla, Water pepper, Arctic butterbur, Parsley, Runner Bean, Lima Bean, Bean, Common Reed, Rough fogfruit, Star Gooseberry, Myrobalan, Round-headed rampion, Indian Pokeberry, American Pokeweed, Bella Sombra, Deer calalu, Aniseed, Burnet Saxifrage, Japanese Red Pine, Mexican Pepperleaf, West African Pepper, Cha-phlu, Queensland grass-cloth plant, Tree lettuce, Chinese Pistache, Terebinth, Water Lettuce, Garden Pea, Buckshorn plantain, Long-leaved Plantain, Broad-leaved Plantain, Himalayan mayapple, Knotweed, Bistort, American Bistort, Alpine bistort, Trifoliate orange, Common purslane, Elephant Bush, Cowslip, Primrose, Kerguelen cabbage, Lungwort, Birch-Leaved Pear, Lesser celandine, Wild radish, Radish, Chinese radish, Raffia palm, French Scorzonera, Meadow beauty, Roseroot, Nikau, Blackcurrant, Seven Sisters Rose, Sorrel, Glasswort, Weeping Willow, Rosegold pussy willow, Saltwort, Land Seaweed, Opposite leaved saltwort, Toothbrush tree, Salad Burnet, Great Burnet, Sassafras, Katuk, Eastern Swamp Saxifrage, Creeping Rockfoil, Tagamina, Spotted golden thistle, Scorzonera, Baikal Skullcap, Chayote, Love-restorer, Spreading stonecrop, Jenny's stonecrop, Rose crown, Livelong, Cassod Tree, Sesame de gazelle, Sésame, Benniseed, West Indian pea, Sesban, Sea Purselane, Palm-grass, Arrowleaf sida, Moss campion, Bladder Campion, Blessed milk thistle, White Mustard, Charlock, London rocket, Hedge mustard, Alexanders, Chinese potato, Field sow-thistle, Spiny-leaved sow thistle, Sow Thistle, Pagoda-tree, Toothache Plant, Spinach, Greater Duck-weed, Otaheite Apple, Yellow mombin, Jocote, Common Chickweed, Natal orange, Sea Blite, Malay apple, Jewels of Opar, Tansy, Dandelion, Fluted gourd, New Zealand Spinach, Portia tree, Pennycress, Common Thyme, Chinese Mahogany, Windmill Palm, Western salsify, Salsify, Goat's Beard, Alsike Clover, Red Clover, White Clover, Sweet Trefoil, Wake-robin, White trillium, Painted trillium, Garden Nasturtium, Dwarf Nasturtium, Mashua, Coltsfoot, Ulluco, Siberian elm, Rose Mallow, Stinging Nettle, Annual Nettle, Italian Corn Salad, Corn Salad, European Verbena, Bitter leaf, Water Speedwell, Brooklime, Canada Violet, Sweet Violet, Bird's Foot Violet, Common blue violet, Amur grape, California wild grape, Northern Fox Grape, Grape, Wasabi, Japanese wisteria, Yellowhorn, and Awapuhi.

### BRIEF DESCRIPTION OF THE DRAWINGS

FIG. 1. Aurone formation in pSIM1251 tobacco. Diagram of the pSIM1251 transfer DNA. B=T-DNA border, P=promoter, T=terminator (A). Flower of transgenic tobacco (B) and untransformed snapdragon (C). HPLC chromatogram of pSIM1251 tobacco (D) and snapdragon flowers (E) showing AOG eluting as peak 1 and 1′ at 400 nm. Mass spectra and MS-MS fragmentation of m/z 449 of AOG from snapdragon (F). mAU, milliabsorbance units.

FIG. 2. Overexpression of StMtf1^{m} in potato. Typical phenotype of 646 tobacco leaves (top) and flowers (bottom) (A). Extracts used to generate HPLC chromatograms were from leaves of untransformed tobacco plants recorded at 520 nm (B) and of 646 plants recorded at 520 nm for anthocyanins (C) and 360 nm for flavonoids (D). Peaks: (2) unidentified anthocyanin at 2.4 min, (3) cyanidin-3-O-glucoside at 2.6 min, (4) pentahydroxy flavone-glucoside at 5.1 min. For quantitative analysis, see Table 1. RT, retention time.

FIG. 3. HPLC chromatograms of 646/1252 tobacco plants. Extracts were obtained from leaves of 646/1252 plants recorded at 520 nm for anthocyanin (A), untransformed plants at 360 nm for flavonoids (B), and 646/1252 plants, 360 nm (C). The UV spectrum of peak 9 is shown in (D). Peaks: (4) pentahydroxy flavone-glucose, (5) naringenin chalcone derivative, (6) naringenin chalcone-diglucose, (7) naringenin chalcone-glucose, (8) tetrahydroxy methoxychalcone-glucose, and (9) naringenin chalcone. Quantitative analyses are summarized in Table 1.

FIG. 4. Aurone formation in transgenic tobacco. Phenotype of a greenhouse-grown 646/1252/1251 plant (A) and individual leaves of controls (B and C, left) and 646/1252/1251 plants (B and C, right). Flowers are shown for control (D), 646 (E), 646/1252 (F) and 646/1252/1257 (G) plants. An HPLC chromatogram of 646/1252/1257 recorded at 520 nm for anthocyanin (H), 360 nm for flavonoids (I) and at 400 nm for aurone (J). Compounds eluting at 2.5 and 3.9 min and denoted as peaks 1 and 1′, respectively, were both identified as AOG and compared to wild-type, 646, 646/1252 and 646/1257 plants (K-N). Mass spectra and MS/MS fragmentation of AOG (m/z=449) in the positive ion mode. (O). Comparison of UV spectra of AOG from snapdragon flower (P) and the 646/1252/1257 plant (Q).

FIG. 5. Aurone production in transgenic lettuce. Leaves of control (left) and two 1610 plants (right) (A-B). HPLC chromatograms of lettuce leaf extracts detected at 400 nm for aurone. Extracts were obtained from leaves of untransformed (C), 1610 (D), 1618(E) and 1610/1618 (F) lettuce plants. Peak 2, which eluted at 4.2 min, was identified as aureusidin-6-O-glucoside. For quantitative data, see Table 2.

FIG. 6. HPLC chromatograms detected at 360 nm for flavonoids. Extracts used were obtained from the leaves of untransformed lettuce (A) and the 1610 (B), 1618 (C) and 1610/1618 (D) plants. The UV spectrum of peak 5 (E) indicates the typical flavonoid λₘₐₓ. Peaks 3, 4 and 5 represent quercetin derivative, kaempferol-glucoside and quercetin-3-(6′-malonyl) glucoside, respectively. For a detailed quantitative analysis, see Table 2.

FIG. 7. T1 seedling of triply transformed (646/1252/1257) tobacco showing various colors due to different gene combinations.

FIG. 8. Comparison of UV-Vis spectra of aurone peaks in snapdragon flower extract and 646/1252/1257 tobacco plants. Peaks (1) eluting at 2.5 min (A) and (1′) eluting at 3.9 min (B) show virtually identical absorption maxima, which is the characteristic UV pattern of aurone. Peak 1′, tentatively identified as an isomer of AOG, eluted at 3.9 min.

FIG. 9. Anthocyanin in deep purple tobacco plants. UV-Vis spectrum of anthocyanin peak 3 at 2.6 min (A) and positive ion mass spectra and MS² fragmentation of m/z 595 (red), identified as cyanidin-3-O-rutinoside (B).

FIG. 10. Flavone accumulated in 646/1252 tobacco plants. Positive ion mass spectra and MS² fragmentation of m/z 435 for naringenin chalcone glucoside (A), m/z 465 for tetrahydroxy methoxychalcone glucoside (B) and m/z 272.9 for naringenin chalcone (C).

FIG. 11. HPLC chromatograms of transgenic lettuce leaf extracts recorded at 520 nm for anthocyanin. Extracts used were from leaves of wild-type (A) 1610 (B) 1618 (C) and 1610/1618 (D) lettuce plants and UV-Vis absorption maxima of peak 2 revealed coelution of aurone and anthocyanin (E). Peak 2, identified as cyanidin 3-(6′-malonyl) glucoside, coeluted with aureusidin-6-O-glucoside. See Table 2 for quantitation.

FIG. 12. Anthocyanin in wild-type and transgenic lettuce plants. Positive ion mass spectra and MS/MS fragmentation of m/z 535.1, identified as cyanidin 3-(6′-malonyl) glucoside (A), and UV-Vis spectrum (B).

FIG. 13. Plasmid map of pSIM1251.

FIG. 14. Plasmid map of pSIM1252.

FIG. 15. Plasmid map of pSIM1257.

FIG. 16. Plasmid map of pSIM1610.

FIG. 17. Plasmid map of pSIM1618.

FIG. 18. Plasmid map of pSIM646.

### DETAILED DESCRIPTION

All references cited in this application are incorporated by references by their entireties.

The health-promoting property of diets rich in fruits and vegetables is based, in part, on the additive and synergistic effects of multiple antioxidants. To further enhance food quality, the capability to synthesize a yellow antioxidant, aureusidin that is normally produced only by some ornamental plants, was introduced into plants. For this purpose, the snapdragon (Antirrhinum majus) chalcone 4′-O-glucosyltransferase (Am4′CGT) and aureusidin synthase (AmAs1) genes, which catalyze the synthesis of aureusidin from chalcone, were expressed in tobacco (Nicotiana tabacum) and lettuce (Lactuca sativa) plants that displayed a functionally active chalcone/flavanone biosynthetic pathway. Leaves of the resulting transgenic plants developed a yellow hue and displayed higher super oxide dismutase (SOD) inhibiting and oxygen radical absorbance capacity (ORAC) activities than control leaves. The results presented herein suggest that the nutritional qualities of leafy vegetables can be enhanced through the introduction of aurone biosynthetic pathways.

### Method for Modifying a Plant

Many embodiments of the present invention relate to a method for modifying a plant, comprising overexpressing or expressing de novo at least one of (i) chalcone 4′-O-glucosyltransferase, and (ii) aureusidin synthase, in the plant.

As described herein, “expressing de novo” means expressing a polypeptide that is not normally expressed in a plant, while “overexpressing” means expressing a polypeptide at a level higher than its normal expression level in a plant.

The method described herein can comprise, for example, overexpressing or expressing de novo chalcone 4′-O-glucosyltransferase in a plant. The chalcone 4′-O-glucosyltransferase can be overexpressed or expressed do novo in, for example, the flowers and/or leaves of the modified plant. The 4′-O-glucosyltransferase gene can be cloned from, for example, an aurone producing plant such as snapdragon, and optionally modified. The chalcone 4′-O-glucosyltransferase can be, for example, Antirrhinum majus chalcone 4′-O-glucosyltransferase (Am4′CGT). In one embodiment, the chalcone 4′-O-glucosyltransferase comprise the DNA sequence of SEQ ID NO:1.

The method described herein can comprise, for example, overexpressing or expressing de novo aureusidin synthase in a plant. The aureusidin synthase can be overexpressed or expressed do novo in, for example, the flowers and/or leaves of the modified plant. The aureusidin synthase gene can be cloned from, for example, an aurone producing plant such as snapdragon, and optionally modified. The aureusidin synthase can be, for example, Antirrhinum majus aureusidin synthase (AmAs1). In one embodiment, the aureusidin synthase comprise the DNA sequence of SEQ ID NO:2.

The method described herein can comprise, for example, overexpressing or expressing de novo both chalcone 4′-O-glucosyltransferase and aureusidin synthase in a plant. Both the chalcone 4′-O-glucosyltransferase and the aureusidin synthase can be overexpressed or expressed do novo in, for example, the flowers and/or leaves of the modified plant. The method described herein can comprise, for example, overexpressing or expressing de novo both Am4′CGT and AmAs1 in a plant.

The de novo expression or overexpression of chalcone 4′-O-glucosyltransferase and/or aureusidin synthase in the modified plant can increase the production of at least one aurone, such as aureusidin-6-O-glucoside. The increased production of aureusidin-6-O-glucoside can be observed in, for example, the flowers of the modified plant. The increased production of aureusidin-6-O-glucoside can be observed in, for example, the leaves of the modified plant. The increased production of aureusidin-6-O-glucoside can be observed in, for example, both the flowers and the leaves of the modified plant. The flowers and/or leaves of the modified plant can develop, for example, a yellow hue.

The method described herein can increase the level of aureusidin-6-O-glucoside production in the leaves of the modified plant by, for example, at least 20%, or at least 50%, or at least 100%, or at least 200%, or at least 500%, or at least 1000%, compared to a wild plant of the same variety. The concentration of aureusidin-6-O-glucoside in the leaves of the modified plant can be, for example, at least 5%, or at least 10%, or at least 20%, or at least 30%, or at least 40%, or at least 50% of the aureusidin-6-O-glucoside concentration in the flowers of a wild plant of Antirrhinum majus.

The method described herein can increase the super oxide dismutase (SOD) inhibiting activities of the leaves of the modified plant by, for example, at least 20%, or at least 40%, or at least 60%, or at least 80%, or at least 100%, compared to a wild plant of the same variety. The method described herein can increase oxygen radical absorbance capacity (ORAC) activities of the leaves of the modified plant by, for example, at least 20%, or at least 40%, or at least 60%, or at least 80%, or at least 100%, compared to a wild plant of the same variety.

In some embodiments, the plant described herein is a dicotyledonous plant. In some embodiments, the plant is a leaf vegetable. In one particular embodiment, the plant is lettuce. In another particular embodiment, the plant is tobacco.

The method described herein can be implemented by, for example, transforming a plant with one or more expression cassettes that express in the plant at least one of the 4′-O-glucosyltransferase gene (e.g., Am4′CGT) and the aureusidin synthase gene (e.g., AmAs1). The method can be implemented by, for example, (A) stably integrating into the genome of at least one plant cell one or more exogenous genetic cassettes selected from the group consisting of (i) a gene expression cassette for expressing 4′-O-glucosyltransferase (e.g., Am4′CGT) and (ii) a gene expression cassette for expressing aureusidin synthase (e.g., AmAs1), and (B) regenerating the transformed plant cell into a plant. In a preferred embodiment, Agrobacterium-mediated transformation is used to produce the transformed plant cell.

The method described herein for producing aureusidin-6-O-glucoside can be further improved by, for example, increasing the production and/or accumulation of naringenin chalcone, the precursor of aureusidin-6-O-glucoside.

To increase the production of naringenin chalcone, one or more genes involved in the biosynthesis of naringenin chalcone can be overexpressed or expressed de novo in the modified plant. In a particular embodiment, potato transcription factor StMtf1^{M} is overexpressed or expressed de novo to activate the flavanoid pathway.

To increase the accumulation of naringenin chalcone, one or more genes involved in the conversion of naringenin chalcone to anthocyanin can be downregulated in the modified plant. In a particular embodiment, chalcone isomerase is downregulated to increase the accumulation of naringenin chalcone. In another particular embodiment, dihydro flavonol 4-reductase is downregulated to increase the accumulation of naringenin chalcone.

In some embodiments, the method described herein comprises (A) overexpressing or expressing de novo both chalcone 4′-O-glucosyltransferase and aureusidin synthase in a plant, and (B) overexpressing or expressing de novo at least one gene involved in the biosynthesis of naringenin chalcone to increase the production of naringenin chalcone.

In some embodiments, the method described herein comprises (A) overexpressing or expressing de novo both chalcone 4′-O-glucosyltransferase and aureusidin synthase in a plant, and (B) downregulating at least one gene involved in the conversion of naringenin chalcone to anthocyanin to decrease the consumption of naringenin chalcone for anthocyanin biosynthesis.

In some embodiments, the method described herein comprises (A) overexpressing or expressing de novo both chalcone 4′-O-glucosyltransferase and aureusidin synthase in a plant, (B) overexpressing or expressing de novo at least one gene involved in the biosynthesis of naringenin chalcone to increase the production of naringenin chalcone, and (C) down-regulating at least one gene involved in the conversion of naringenin chalcone to anthocyanin to decrease the consumption of naringenin chalcone for anthocyanin biosynthesis.

In some embodiments, the method described herein comprises (A) overexpressing or expressing de novo at least one gene involved in the biosynthesis of naringenin chalcone to increase the production of naringenin chalcone, and (B) downregulating at least one gene involved in the conversion of naringenin chalcone to anthocyanin to decrease the consumption of naringenin chalcone for anthocyanin biosynthesis.

In some embodiments, the method described herein comprises (A) overexpressing or expressing de novo both chalcone 4′-O-glucosyltransferase and aureusidin synthase in a plant, (B) measuring the level of aureusidin-6-O-glucoside in the modified plant, and optionally (C) overexpressing or expressing de novo at least one gene involved in the biosynthesis of naringenin chalcone and/or downregulating at least one gene involved in the conversion of naringenin chalcone to anthocyanin, so as to further boost the level of aureusidin-6-O-glucoside in the modified plant.

The method described herein can further comprise, for example, extracting aurone, such as aureusidin-6-O-glucoside, from the modified plant. The method described herein can further comprise, for example, incorporating the leaves of the modified plant or the aurone extracted therefrom into a food product or a nutritional composition.

### Transformation Vectors

Many embodiments of the present invention also relate to one or more transformation vectors for transforming plant cells. The transformation vector can comprise, for example, one or more expression cassettes selected from the group consisting of (i) a gene expression cassette for expressing the chalcone 4′-O-glucosyltransferase gene, and (ii) a gene expression cassette for expressing the aureusidin synthase gene.

The transformation vector can be, for example, a binary vector suitable for Agrobacterium-mediated transformation. See, e.g., Komori et al., Plant Physiology 145:1155-1160 (2007) and Hellens et al., Trends in Plant Science 5(10):446-451 (2000), incorporated herein by reference in their entireties. The binary vector can comprise, for example, a transfer DNA region delineated by two T-DNA border or plant-derived border-like sequences, wherein the expression cassettes described herein is located in the transfer DNA region. See USP 2012/0297500, incorporated herein by reference in its entirety.

Agrobacterium stains suitable for transforming binary vectors are known in the art and described in, for example, Lee et al., Plant Physiology 146:325-332 (2008), incorporated herein by reference in its entirety. In one particular embodiment, the Agrobacterium stain used for harboring the transformation vector is LBA4404. In another particular embodiment, the Agrobacterium stain used for harboring the transformation vector is AGL-1.

The transformation vector can comprise, for example, a gene expression cassette for expressing the chalcone 4′-O-glucosyltransferase gene (e.g., Am4′CGT). The expression cassette can comprise, from 5′ to 3′, (i) a promoter functional in a plant cell, operably linked to (ii) at least one copy the chalcone 4′-O-glucosyltransferase gene or fragment thereof, and (iii) a terminator functional in a plant cell. The promoter can be, for example, functional in the leaves of the plant. The promoter can be, for example, a leaf-specific promoter.

The transformation vector can comprise, for example, a gene expression cassette for expressing the aureusidin synthase gene (e.g., AmAs1). The expression cassette can comprise, from 5′ to 3′, (i) a promoter functional in a plant cell, operably linked to (ii) at least one copy the aureusidin synthase gene or fragment thereof, and (iii) a terminator functional in a plant cell. The promoter can be, for example, functional in the leaves of the plant. The promoter can be, for example, a leaf-specific promoter.

The transformation vector can comprise, for example, two or more gene expression cassettes. The transformation vector can comprise, for example, a first gene expression cassette for expressing the chalcone 4′-O-glucosyltransferase gene, and a second gene expression cassette for expressing the aureusidin synthase gene.

The transformation vector can further comprise, for example, a gene expression cassette for expressing at least one gene involved in the biosynthesis of naringenin chalcone. The transformation vector can further comprise, for example, a gene silencing cassette for downregulating at least one gene involved in the conversion of naringenin chalcone to anthocyanin, such as chalcone isomerase and/or dihydro flavonol 4-reductase.

### Modified Plants

Many embodiments of the present invention also relate to a modified plant comprising in its genome one or more exogenous genetic cassettes selected from the group consisting of (i) a gene expression cassette for expressing chalcone 4′-O-glucosyltransferase, and (ii) a gene expression cassette for expressing aureusidin synthase.

The modified plant described herein can comprise an inserted chalcone 4′-O-glucosyltransferase gene expression cassette and have, for example, increased production of aurone, such as aureusidin-6-O-glucoside, in its flowers and/or leaves. The chalcone 4′-O-glucosyltransferase gene can be cloned from, for example, an aurone producing plant such as snapdragon, and optionally modified. The chalcone 4′-O-glucosyltransferase can be, for example, Antirrhinum majus chalcone 4′-O-glucosyltransferase (Am4′CGT). In one embodiment, the chalcone 4′-O-glucosyltransferase comprise the DNA sequence of SEQ ID NO:1.

The modified plant described herein can comprise an inserted chalcone aureusidin synthase gene expression cassette and have, for example, increased production of aurone, such as aureusidin-6-O-glucoside, in its flowers and/or leaves. The aureusidin synthase gene can be cloned from, for example, an aurone producing plant such as snapdragon, and optionally modified. The aureusidin synthase can be, for example, Antirrhinum majus aureusidin synthase (AmAs1). In one embodiment, the aureusidin synthase comprise the DNA sequence of SEQ ID NO:2.

The modified plant can have increased production of at least one aurone, such as aureusidin-6-O-glucoside. The increased production of aureusidin-6-O-glucoside can be observed in, for example, the flowers of the modified plant. The increased production of aureusidin-6-O-glucoside can be observed in, for example, the leaves of the modified plant. The increased production of aureusidin-6-O-glucoside can be observed in both the flowers and the leaves of the modified plant.

The modified plant described herein can produce, for example, at least 20% more, or at least 50% more, or at least 100% more, or at least 200% more, or at least 500% more, or at least 1000% more aureusidin-6-O-glucoside than a wild plant of the same variety. The concentration of aureusidin-6-O-glucoside in the leaves of the modified plant can be, for example, at least 5%, or at least 10%, or at least 20%, or at least 30%, or at least 40%, or at least 50% of the aureusidin-6-O-glucoside concentration in the flowers of a wild plant of Antirrhinum majus.

The leaves of the modified plant described herein can have, for example, super oxide dismutase (SOD) inhibiting activities that are at least 20% more, or at least 40% more, or at least 60% more, or at least 80% more, or at least 100% more than a wild plant of the same variety. The leaves of the modified plant described herein can have, for example, oxygen radical absorbance capacity (ORAC) activities that are at least 20% more, or at least 40% more, or at least 60% more, or at least 80% more, or at least 100% more than a wild plant of the same variety.

The modified plant described herein can have, for example, altered color. The flowers of the modified plant can be yellower than the flowers of a wild plant of the same variety. The leaves of the modified plant can be yellower than the leaves of a wild plant of the same variety.

In some embodiments, the modified plant described herein is a dicotyledonous plant. In some embodiments, the modified plant is a leaf vegetable. In one particular embodiment, the modified plant is lettuce. In another particular embodiment, the modified plant is tobacco.

### Food Products

Further embodiments relate to food products and/or nutritional compositions produced from the modified plants described herein. The food product and/or nutritional composition can be made from, for example, the leaves and/or flowers of the modified plant. Compare to food products made from a wild plant of the same variety, the food product described herein can have enhanced antioxidant effect.

### Additional Embodiments

### Embodiment 1

A method for modifying a plant, comprising overexpressing or expressing de novo at least one of (i) chalcone 4′-O-glucosyltransferase, and (ii) aureusidin synthase, in the plant.

### Embodiment 2

The method of Embodiment 1, comprising expressing de novo or overexpressing chalcone 4′-O-glucosyltransferase in the flowers and/or leaves of said plant.

### Embodiment 3

The method of Embodiment 1 or 2, comprising expressing de novo or overexpressing aureusidin synthase in the flowers and/or leaves said plant.

### Embodiment 4

The method of any of Embodiment 1-3, comprising expressing de novo or overexpressing Antirrhinum majus chalcone 4′-O-glucosyltransferase (Am4′CGT) in a plant other than Antirrhinum majus.

### Embodiment 5

The method of any of Embodiments 1-4, comprising expressing de novo or overexpressing Antirrhinum majus aureusidin synthase (AmAs1) in a plant other than Antirrhinum majus.

### Embodiment 6

The method of any of Embodiment 1-5, wherein the chalcone 4′-O-glucosyltransferase gene either comprises the DNA sequence of SEQ ID NO:1, or encodes the protein of SEQ ID NO:2; and wherein the aureusidin synthase gene either comprises the DNA sequence of SEQ ID NO:3, or encodes the protein of SEQ ID NO:4.

### Embodiment 7

The method of any of Embodiment 1-6, comprising transforming a plant with one or more expression cassettes that express at least one of chalcone 4′-O-glucosyltransferase and aureusidin synthase.

### Embodiment 8

The method of any of Embodiment 1-7, comprising (A) stably integrating into the genome of at least one plant cell one or more exogenous genetic cassettes selected from the group consisting of (i) a gene expression cassette for expressing chalcone 4′-O-glucosyltransferase, and (ii) a gene expression cassette for expressing aureusidin synthase; and (B) regenerating the transformed plant cell into a plant.

### Embodiment 9

The method of any of Embodiment 1-8, further comprising overexpressing or expressing de novo one or more genes involved in the biosynthesis of naringenin chalcone in order to increase the production of naringenin chalcone in said plant.

### Embodiment 10

The method of any of Embodiment 1-9, further comprising downregulating one or more genes involved in the conversion of naringenin chalcone to anthocyanin, such as chalcone isomerase and dihydro flavonol 4-reductase, in order to decrease the consumption of naringenin chalcone for anthocyanin biosynthesis.

### Embodiment 11

The method of any of Embodiment 1-10, comprising (A) overexpressing or expressing de novo both chalcone 4′-O-glucosyltransferase and aureusidin synthase in the plant, (B) measuring the level of aureusidin-6-O-glucoside in the modified plant, and optionally (C) overexpressing or expressing de novo at least one gene involved in the biosynthesis of naringenin chalcone and/or downregulating at least one gene involved in the conversion of naringenin chalcone to anthocyanin, so as to further boost the level of aureusidin-6-O-glucoside in the plant.

### Embodiment 12

The method of any of Embodiment 1-11, wherein the leaves of said plant produces at least 50% more, at least 100% more, or at least 200% more aureusidin-6-O-glucoside than the leaves of a wild plant of the same variety.

### Embodiment 13

The method of any of Embodiment 1-12, wherein the concentration of aureusidin-6-O-glucoside in the leaves of said plant is at least 10%, at least 20%, or at least 30% of the aureusidin-6-O-glucoside concentration in the flowers of a wild plant of Antirrhinum majus.

### Embodiment 14

The method of any of Embodiment 1-13, wherein the leaves said plant display super oxide dismutase (SOD) inhibiting activities that are at least 20% more, or at least 40% more, or at least 60% more, or at least 80% more, or at least 100% more than the leaves of a wild plant of the same variety.

### Embodiment 15

The method of any of Embodiment 1-14, wherein the leaves of said plant display oxygen radical absorbance capacity (ORAC) activities that are at least 20% more, or at least 40% more, or at least 60% more, or at least 80% more, or at least 100% more than the leaves of a wild plant of the same variety.

### Embodiment 16

The method of any of Embodiment 1-15, wherein said plant is leaf plant such as tobacco or lettuce.

### Embodiment 17

A modified plant made according to the method of any of Embodiments 1-16.

### Embodiment 18

A modified plant comprising in its genome one or more exogenous genetic cassettes selected from the group consisting of (i) a gene expression cassette for expressing the chalcone 4′-O-glucosyltransferase gene, and (ii) a gene expression cassette for expressing the aureusidin synthase gene.

### Embodiment 19

The plant of Embodiment 18, comprising both the chalcone 4′-O-glucosyltransferase gene expression cassette and the aureusidin synthase gene expression cassette.

### Embodiment 20

The plant of any of Embodiment 18-19, wherein chalcone 4′-O-glucosyltransferase is overexpressed or expressed de novo in the flowers and/or leaves of said plant.

### Embodiment 21

The plant of any of Embodiment 18-20, wherein aureusidin synthase is overexpressed or expressed de novo in the flowers and/or leaves of said plant.

### Embodiment 22

The plant of any of Embodiment 18-21, wherein the chalcone 4′-O-glucosyltransferase gene and the aureusidin synthase gene are cloned from Antirrhinum majus and optionally modified.

### Embodiment 23

The plant of any of Embodiment 18-22, wherein said plant is leaf plant such as tobacco or lettuce.

### Embodiment 24

The plant of any of Embodiment 18-23, wherein the leaves of said plant produces at least 50% more, or at least 100% more, or at least 200% more aureusidin-6-O-glucoside than the leaves of a wild plant of the same variety.

### Embodiment 25

The plant of any of Embodiment 18-24, wherein the concentration of aureusidin-6-O-glucoside in the leaves of said plant is at least 10%, or at least 20%, or at least 30% of the aureusidin-6-O-glucoside concentration in the flowers of a wild plant of Antirrhinum majus.

### Embodiment 26

The plant of any of Embodiment 18-25, wherein the leaves said plant display super oxide dismutase (SOD) inhibiting activities that are at least 20% more, or at least 40% more, or at least 60% more, or at least 80% more, or at least 100% more than the leaves of a wild plant of the same variety.

### Embodiment 27

The plant of any of Embodiment 18-26, wherein the leaves of said plant display oxygen radical absorbance capacity (ORAC) activities that are at least 20% more, or at least 40% more, or at least 60% more, or at least 80% more, or at least 100% more than the leaves of a wild plant of the same variety.

### Embodiment 28

A food product or nutritional supplement produced from the plant of any of Embodiment 17-27.

### Embodiment 29

A plant transformation vector, comprising one or more genetic cassettes selected from the group consisting of (i) a gene expression cassette for expressing the chalcone 4′-O-glucosyltransferase gene, and (ii) a gene expression cassette for expressing the aureusidin synthase gene.

### Embodiment 30

The method of any of Embodiment 1-16, further comprising overexpressing or expressing de novo potato transcription factor StMtf1^{M} in said plant.

### Embodiment 31

A method for increase the accumulation of naringenin chalcone in a plant, comprising downregulating chalcone isomerase and/or dihydro flavonol 4-reductase in said plant.

### Embodiment 32

A method for increase the availability of naringenin chalcone for aurone production in a plant, comprising (A) overexpressing or expressing de novo potato transcription factor StMtf1^{M} in said plant, and (B) downregulating chalcone isomerase and/or dihydro flavonol 4-reductase in said plant.

### Embodiment 33

A method comprising: (A) stably integrating into the genome of at least one plant cell (i) an exogenous gene expression cassette for expressing chalcone 4′-O-glucosyltransferase and (ii) an exogenous gene expression cassette for expressing aureusidin synthase, and (B) proliferating the transformed plant cell in the presence of naringenin chalcone.

### EXAMPLES

### Example 1

### Methods and Materials

Chemicals and Standards.

HPLC grade acetonitrile, water and trifluoroacetic acid (TFA) and also naringenin and chalcone standards were purchased from Sigma (St. Louis, Mo., USA). Naringenin-7-O-rutinoside and cyanidin-3-O-glucoside were purchased from Indofine (Hillsborough, N.J.). Maritimein (3′,4′,6,7-tetrahydroxyaurone or maritimetin-7-glucoside) was purchased from Chromadex (Irvine, Calif.). All standards were prepared as stock solutions at 10 mg/mL in methanol and diluted in water, except for chalcone, which was prepared in 50% methanol. UV external standard calibration was used to obtain calibration curves of cyanidin-3-O-glucose, naringenin-7-O-rutinoside, and chalcone, which were used to quantify anthocyanins, flavones, and chalcones, respectively. Both UV and mass spectrometry (MS) external calibration of maritimein were employed for quantitation of aureusidin-6-O-glucose.

Genes and Plasmid Constructs.

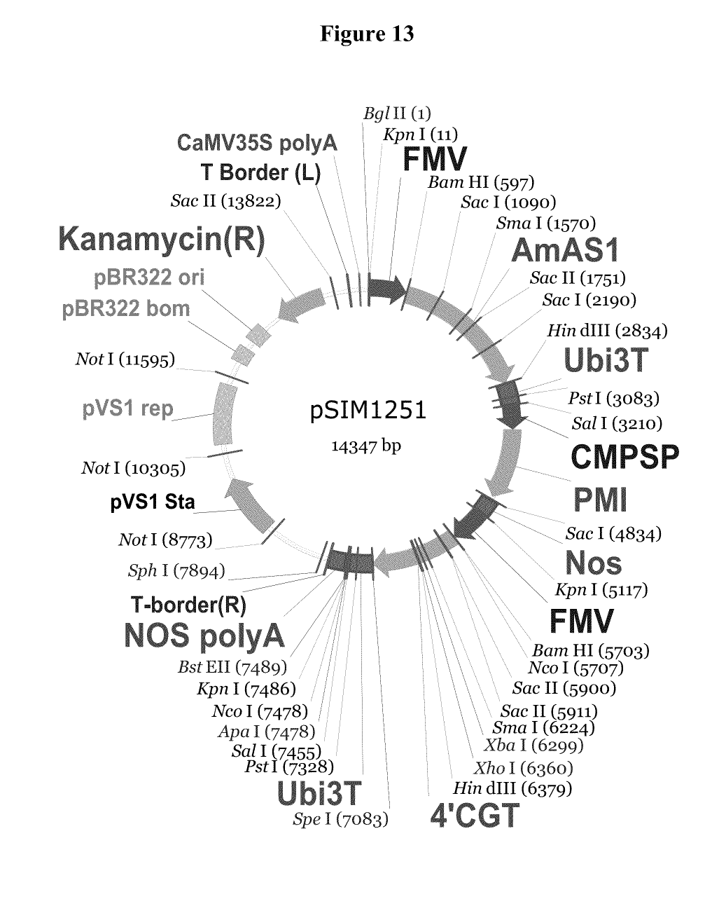

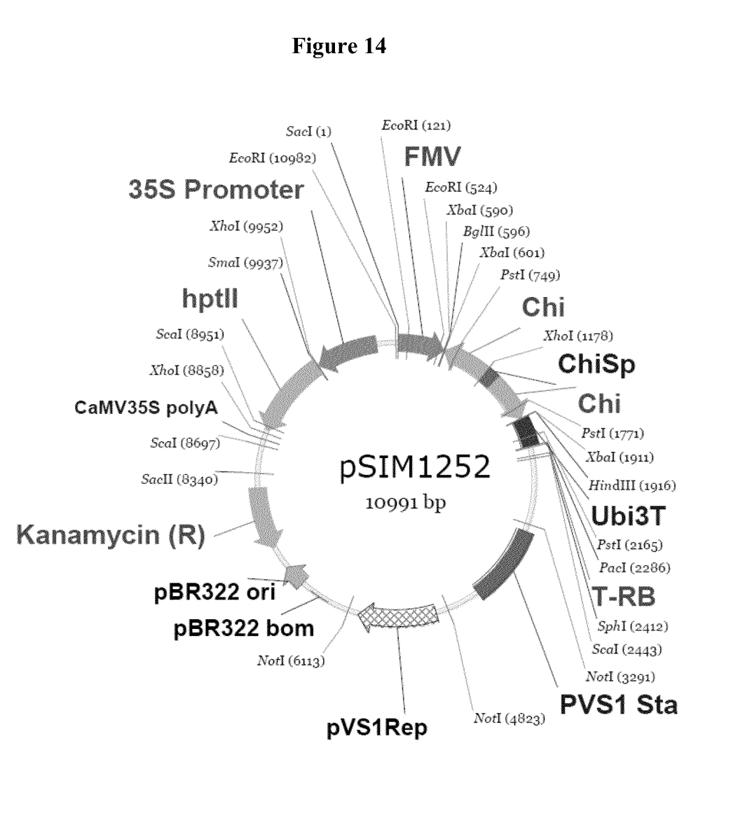

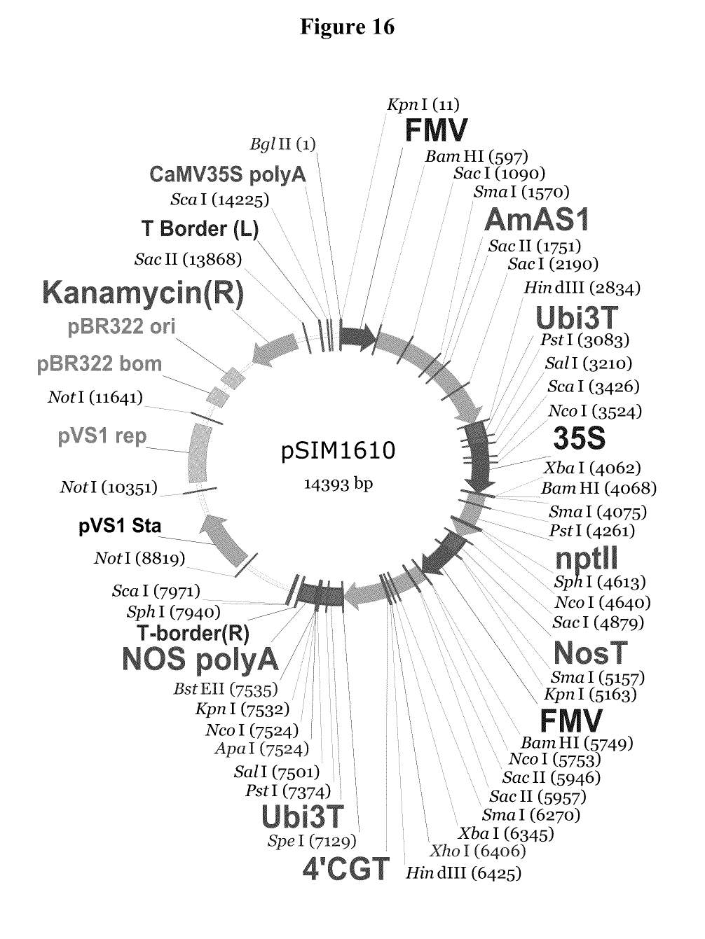

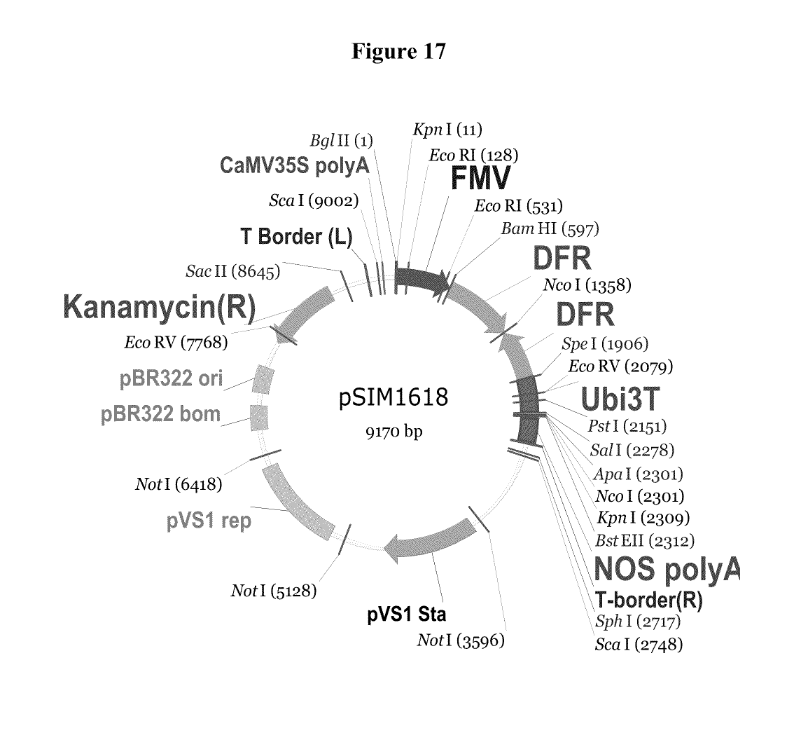

A full-length cDNA of the aureusidin synthase (AmAS1) gene (SEQ ID NO:1) was isolated from snapdragon (Antirrhinum majus “Rocket Yellow”) flowers by reverse transcriptase (RT-)PCR using the primer set 5′-GGA TCC AAA TTA CAT TGC TTC CTT TGT CCC AC (forward) and 5′-AAG CTT CTC AAA AAG TAA TCC TTA TTT CAC (reverse). The product digested with BamHI and HindIII was fused to regulatory elements, the 35S promoter of figwort mosaic virus (FMV) and the terminator of the potato ubiquitin-3 gene, and the resulting expression cassette was cloned into pBluescript (Agilent Technologies, Santa Clara, Calif.). The cytosolic chalcone 4′-O-glucosyltransferase (Am4′-Cgt) cDNA (SEQ ID NO:2) was also amplified from flower RNA, and the primer set used in this case was 5′-GGA TCC ATG GGA GAA GAA TAC AAG AAA ACA C (forward) and 5′-ACT AGT TTA ACG AGT GAC CGA GTT GAT G (reverse). The BamHI-HindIII fragment was linked to the FMV promoter and Ubi3 terminator, and also inserted into pBluescript. The binary vector pSIM1251 (FIG. 13) contains both the AmAS1 and Am4′CGT gene expression cassettes and a cassette for the phosphomannose isomerase (pmi) selectable marker gene (Aswath et al., 2006). Vector pSIM1610 (FIG. 16) is similar to pSIM1251, but carries a neomycin phosphotransferase (nptII) selectable marker gene. Primers used to amplify a 0.6-kb fragment of the tobacco chalcone isomerase (Chi) gene (Genbank accession AB213651) had the sequences 5′AGA TCT CTA GAC TCC AAT TTC TGG AAT GGT AG (forward) and 5′-CTC GAG AGT GCT CTT CCT TTT CTC GCC GC (reverse) for the antisense fragment (SEQ ID NO:4), and 5′-CTC GAG GAG TCC ATT ACC ATT GAG AAT TAC G (forward) and 5′-CTC GAG GAG TCC ATT ACC ATT GAG AAT TAC G (reverse) for the sense counterpart (SEQ ID NO:3). Vector pSIM1252 (FIG. 14) carries the inverted repeat of Chi gene fragments positioned between the FMV promoter and Ubi3 terminator. A silencing cassette targeting the dihydroflavonol 4-reductase (Dfr) gene from the lettuce variety Eruption (identical to Genbank CV700105) was generated using the primer pairs 5′-GGA TCC GCA GGT ACA ACT AGA CAC CG (forward) and 5′-CCA TGG ATT GGT GTT TAC ATC CTC TGC G (reverse) for a 708-bp sense fragment (SEQ ID NO:5), and 5′-ACT AGT GCA GGT ACA ACT AGA CAC CG (forward) and 5′-CCA TGG AGT CGT TGG TCC ATT CAT CA (reverse) for a 542-bp antisense fragment (SEQ ID NO:6). The vector carrying the inverted repeat of Dfr fragments fused to regulatory elements and positioned within the T-DNA was named pSIM1618 (FIG. 17).

Plant Transformation.

Tobacco was transformed as described previously (Richael et al., 2008). For transformation of the lettuce variety Eruption, ^{˜}250 seeds were transferred to a 1.7-ml Eppendorf tube, immersed for 1 min in 70% ethanol and for 15 min in 10% bleach with a trace of Tween, and then triply rinsed with sterile water. Sterilized seeds were spread evenly over solidified medium consisting of half-strength MS with vitamins (M404, Phytotechnology) containing 10 g sucrose per liter and 2% Gelrite in Magenta boxes (30-40 seeds/box), and germinated at 24° C. under a 16-h day/8-h night regime. Agrobacterium was grown overnight from frozen glycerol stock (−80° C.) in a small volume of Luria Broth with kanamycin (100 mg/L) and streptomycin (100 mg/L). Cotyledons from 4-day old seedlings were wounded with a scalpel to give small cuts at right angles to the midvein, and immersed in Agrobacterium suspensions. After 10 min, the suspension was removed by aspiration and the explants were blotted on sterile filter paper. Explants were placed adaxially on co-culture medium that consisted of MS medium (pH 5.7) with vitamins (M404, Phytotechnology), 30 g sucrose per liter, 0.1 mg/L 6-benzylaminopurine (BAP), and 0.1 mg/L 1-naphthaleneacetic acid (NAA), solidified with 6 g/L agar. After two days, the explants were transferred to regeneration medium that consisted of MS medium (pH 5.7) with vitamins (M404), 30 g sucrose per liter, 0.1 mg/L BAP, 0.1 mg/L NAA, 6 g/L agar, 150 mg/L timentin, and 100 mg/L kanamycin. Explants were transferred to fresh media at 2-week intervals. After 2-3 weeks, shoot buds were harvested and transferred to the same media. Shoots that elongated within the next 2-4 weeks were transferred to media lacking hormones, to promote root formation.

Sample Preparation for Biochemical Analysis.

Greenhouse-grown lettuce or tobacco leaves or flowers were harvested, immediately frozen in liquid N₂ and then homogenized. The powder was then freeze dried and stored at −80° C. until used. Samples were extracted as described by Ono et al., 2006, with modification. Briefly, about 150 mg freeze-dried ground leaves or flowers were placed in a 2-mL screwcap tube along with 50% acetonitrile/0.1% TFA and 500 mg of 1.0-mm glass beads. Tubes were shaken in a BeadBeater (Biospec Bartelsville, Okla.) using a pre-chilled rack for 10 min at maximum speed and centrifuged for 5 min at 4° C., and the supernatant was transferred to a clean tube. The remaining pellet was re-extracted with 1 mL of the same extraction solvent and centrifuged. The supernatants were combined and concentrated in a SpeedVac (Thermo Savant, Waltham, Mo.) prior to HPLC analysis.

In order to confirm anthocyanin, freeze dried leaves were also extracted in acidified methanol (0.01% HCl) for anthocyanin and purified by solid phase extraction using C-18 cartridge as described in Current Protocols in Food Analytical Chemistry (Rodriguez-Saona and Wrolstad, 2001).

LC/MS analysis.

Aurone analyses were performed using an Agilent HPLC series 1200 equipped with ChemStation software, a degasser, quaternary pumps, autosampler with chiller, column oven, and diode-array detector. The separation was performed with an Agilent Zorbax Eclipse XDB-C18 (150×4.6 mm, 5-μm particle size) with a C18 guard column operated at a temperature of 35° C. The mobile phase consisted of 0.1% TFA/water (eluent A) and 90% acetonitrile in water/0.1% TFA (eluent B) at a flow of 0.8 mL/min using the following gradient program: 20% B (0-3 min); 20-60% B (3-20 min); 60% B isocratic (20-27 min); 60-90% B washing step (27-30 min); and equilibration for 10 min. The total run time was 40 min. The injection volume for all samples was 10 μl. Specific wavelengths were monitored separately at 400 nm for aurone and 360 nm for flavones. Additionally, UV/Vis spectra were recorded at 520 nm for anthocyanins. The HPLC system was coupled online to a Bruker (Bremen, Germany) ion trap mass spectrometer fitted with an ESI source. Data acquisition and processing were performed using Bruker software. The mass spectrometer was operated in positive ion mode and auto MSⁿ with a scan range from m/z 100 to 1000. Nitrogen was used both as drying gas at a flow rate of 12 L/min and as nebulizer gas at a pressure of 45 psi. The nebulizer temperature was set at 350° C.

Antioxidant Capacity Assays.

The capacity to scavenge peroxyl and superoxide radicals was determined using 2,2′-azobis (2-amidino-propane) dihydrochloride (AAPH) (Prior et al., 2003; Huang et al., 2005) and a Superoxide Dismutase Activity Assay Kit (BioVision Research Products, Mountain View, Calif.), according to the manufacturer's recommendations. Inhibition of superoxide dismutase was also assayed using the SOD Assay Kit form Cell Technology Company.

### Example 2

### Constitutive Expression of the Snapdragon Am4′CGT and AmAs1 Genes Triggers Flower-Specific Aureusidin Formation in Tobacco

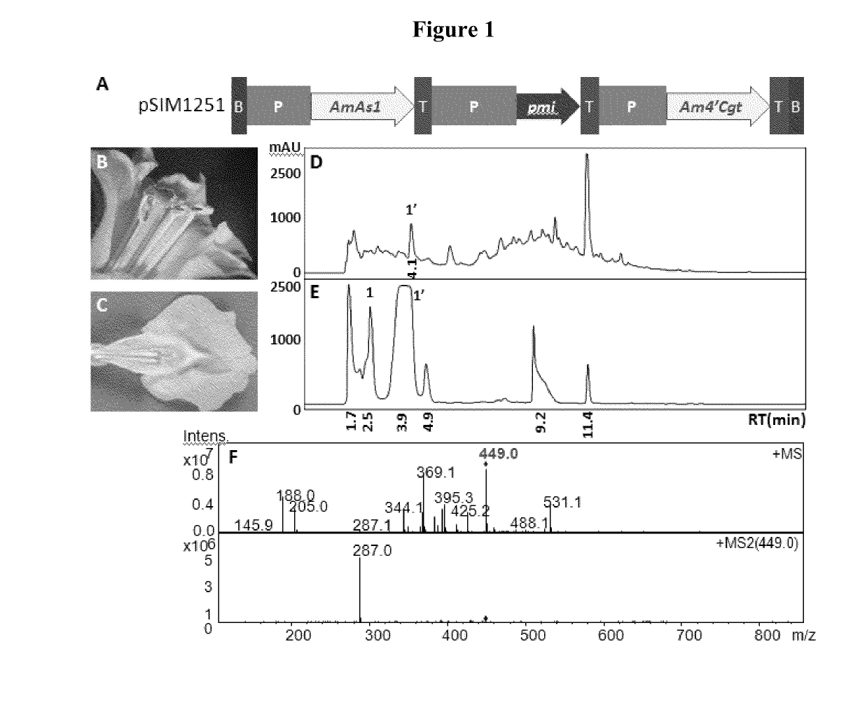

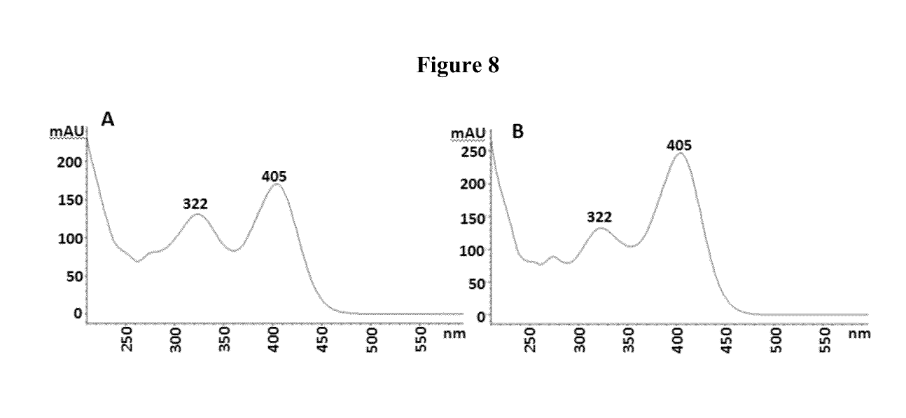

The two snapdragon genes that catalyze aureusidin biosynthesis, Am4′CGT and AmAs1, were operably linked to the strong near-constitutive promoter of figwort mosaic virus (FMV). Insertion of the resulting expression cassettes into the T-DNA of a vector carrying the phosphomannose isomerase (pmi) gene yielded pSIM1251 (FIG. 1A). Agrobacterium-mediated transformation of tobacco (Nicotiana tabacum) produced 25 mannose-resistant plants that, upon PCR-based confirmation of the presence of the three transgenes, were propagated to produce pSIM1251 lines. The original vector carrying only a marker gene was used to generate transgenic controls. One plant of each line was transferred to the greenhouse and allowed to mature at a constant temperature of 28±2° C. Experimental lines appeared phenotypically similar to their transgenic controls, except for flower color. This new color was unusual for tobacco but resembled that of flowers of the untransformed snapdragon variety “Rocket Yellow” used as gene source (FIG. 1B-C). HPLC analysis demonstrated that the yellow transgenic flowers contained a compound that is not naturally produced in tobacco (FIG. 1D, peak 1′). This compound was confirmed to have the same retention time and mass as the predominant flavonoid of snapdragon flowers, which is aureusidin-6-O-glucoside (also named 4,6,3′4′-tetrahydroxyaurone-6-O-glucoside, AOG) (FIG. 1E, peak 1′, and Table 1). MS/MS analysis of peak 1′, which exhibited an [M+H]− ion at m/z 449, yielded MS² fragmentation at m/z 287 due to loss of 162 atomic mass units (amu), corresponding to one glucose moiety (FIG. 1F). In snapdragon, a trace amount of molecular ion m/z 465 was revealed to co-elute with broad peak 1′, which was fragmented at m/z 287 (data not shown) and tentatively identified as bracteatin-6-O-glucoside. Additionally, the mass spectra and UV-Vis features of peak 1, a compound identified in snapdragon but not in the transgenic tobacco, were identical to those of peak 1′ (FIG. 8A-B) and corresponded to an isomer of AOG. Our results demonstrate that the ability to produce AOG can be transferred across family boundaries, from a scrophulariaceous to a solanaceous plant species, through heterologous expression of genes involved in the last two biosynthetic steps. The leaves of pSIM1251 tobacco plants did not contain detectable levels of AOG, indicating that the gene transfer had not broadened the tissue specificity of aurone formation beyond that of snapdragon.

### Example 3

### Chalcone Accumulation Promotes Aureusidin Formation in the Leaves and Stems of Transgenic Tobacco Plants

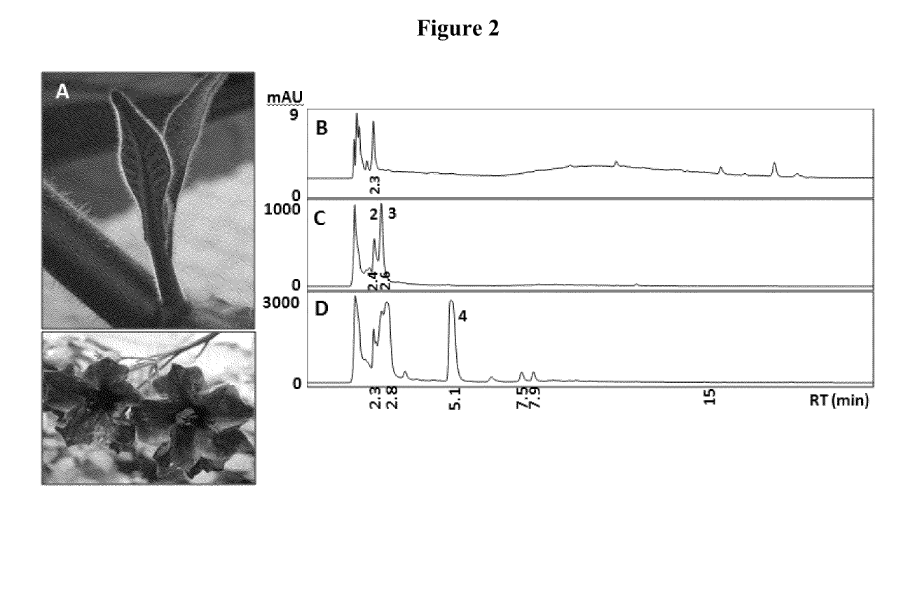

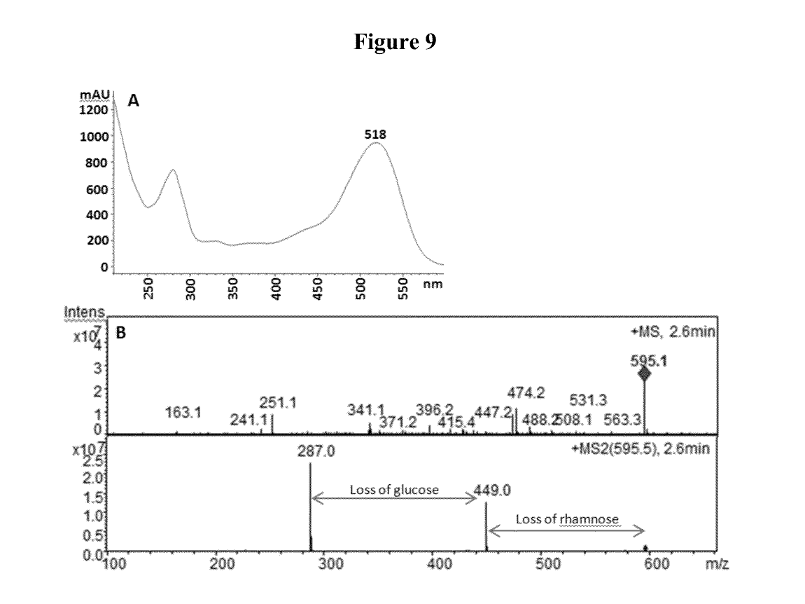

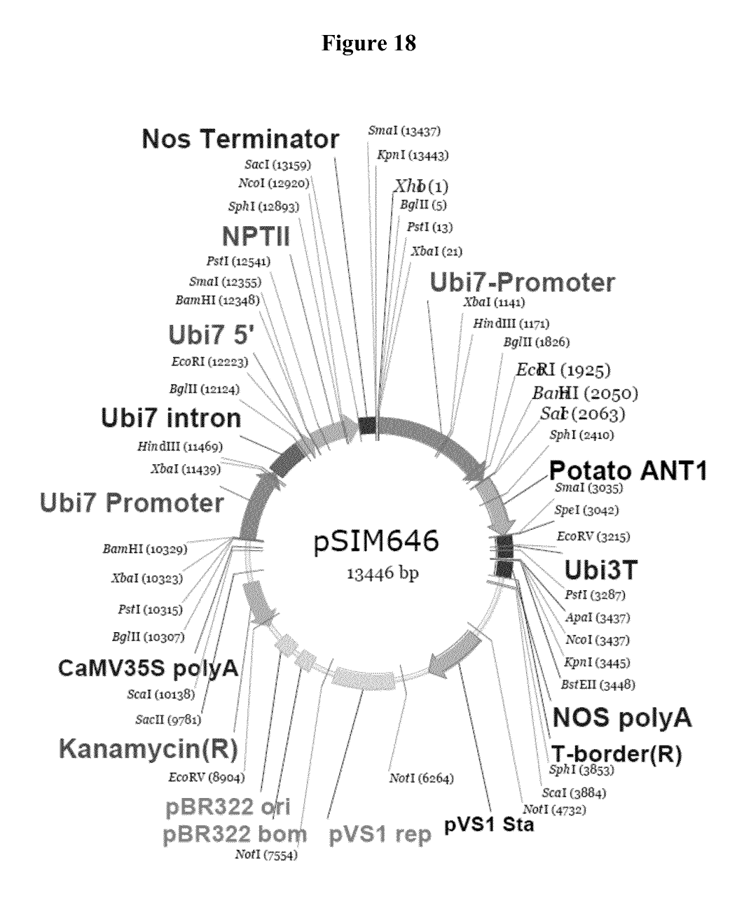

A modified strategy was employed to overcome the flower-limited formation of AOG in tobacco. As a first step, to promote the formation of flavonoid AOG precursors, wild-type tobacco plants were transformed with pSIM646 (FIG. 18). This vector contains the potato (Solanum tuberosum) transcription factor StMtf1^{M} gene (SEQ ID NO:7) fused to the strong promoter of the potato Ubi7 gene (Rommens et al., 2008). The resulting overexpression of the anthocyanin-associated StMtf1^{M} gene produced deep-purple transgenic plants (646 tobacco; FIG. 2A), which were demonstrated by LC/MS to contain large amounts of anthocyanins. Two compounds were not fully separated by LC (FIG. 2B-C, peaks 2 and 3) and had absorption maxima at 518 nm. Using UV-Vis spectra and MS fragmentation, peak 2 was tentatively identified as pelargonidin aglycon (molecular ion at m/z 271, see FIG. 9A), and peak 3 was identified as cyaniding-3-O-rutinose (molecular ion at m/z 595), which could be fragmented to m/z 499 (loss of a rhamnose moiety, 146 amu), and 287 (loss of rutinose, 308 amu) (Table 1 and FIG. 9B). Furthermore, concentrations of a pentahydroxy flavone-glucose, tentatively identified as quercetin-3-O-glucoside, were higher in the StMtf1^{M} plants than in their transgenic controls (FIG. 2D, peak 4; Table 1). The mass spectra of peak 4 at 5.1 min showed a molecular ion at m/z 465 and the MS/MS fragment at m/z 272.9 (data not shown).

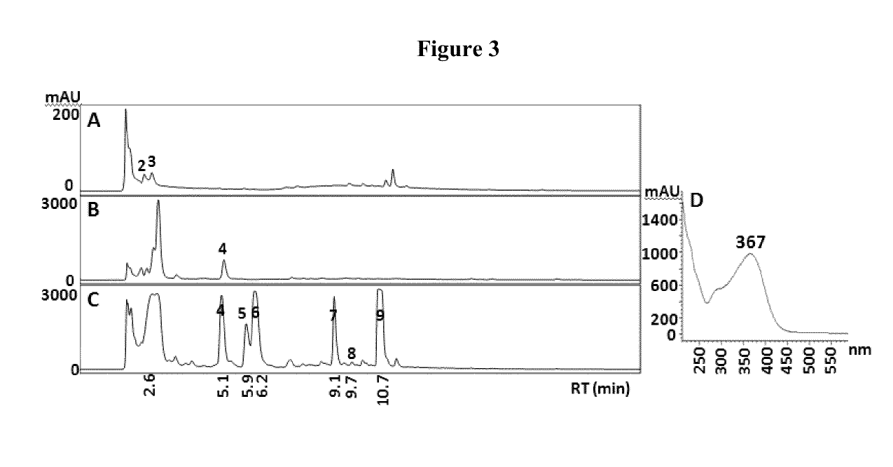

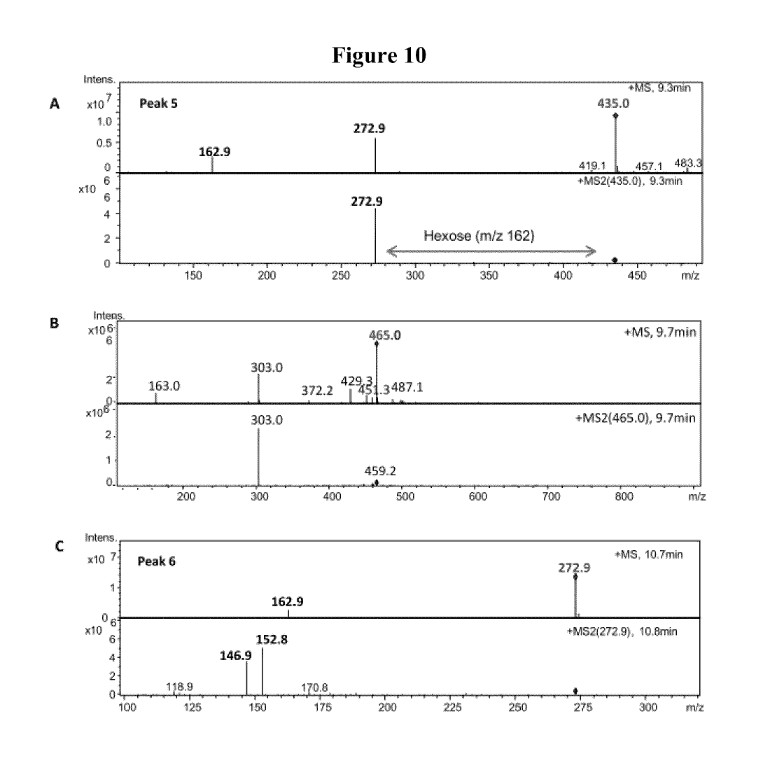

To partially suppress anthocyanin formation and, instead, promote the accumulation of flavonoid intermediates, pSIM646 plants were retransformed with pSIM1252 T-DNA, which carries a silencing cassette targeting the chalcone isomerase (Chi) gene. This second modification altered plant color from deep purple to green with a slight purple hue. The 646/1252 plants accumulated naringenin chalcone and several glycosylated naringenin chalcones. The HPLC chromatograms of anthocyanin and flavonoid profiles are shown in FIG. 3A-C and the quantitative amounts are presented in Table 1. The presence of naringenin chalcone and its glycosylated derivatives was also investigated by MS/MS analysis. The positive ion electrospray product ion tandem mass spectra of m/z 435, 465 and 272.9 are shown in FIG. 10A-C. Peak 5, eluting at 6 min, showed a molecular ion at m/z 272.9, which corresponds to aglycone naringenin chalcone, but no confirmed parent molecular ion was detected. Peak 6, eluting at 6.2 min, was tentatively identified as naringenin chalcone diglucoside with m/z=597 and MS² ion at 272.9 due to loss of 324 amu, corresponding to two glucose moieties. Peak 7, eluting at 9.1 min with a [M+] peak at 435 and a fragment of 272.9 obtained after loss of 162 amu (hexose moiety), was identified as naringenin chalcone glucoside. Peak 8, eluting at 9.7 min with an m/z of 465 and MS² fragment of 303 due to loss of glucose (−162 amu) was attributed to tetrahydroxy methoxychalcone glucoside. Peak 9, eluting at 10.7 min, was identified as naringenin chalcone, according to the mass spectrum with an m/z of 272.9 and UV absorption maximum (FIG. 3D).

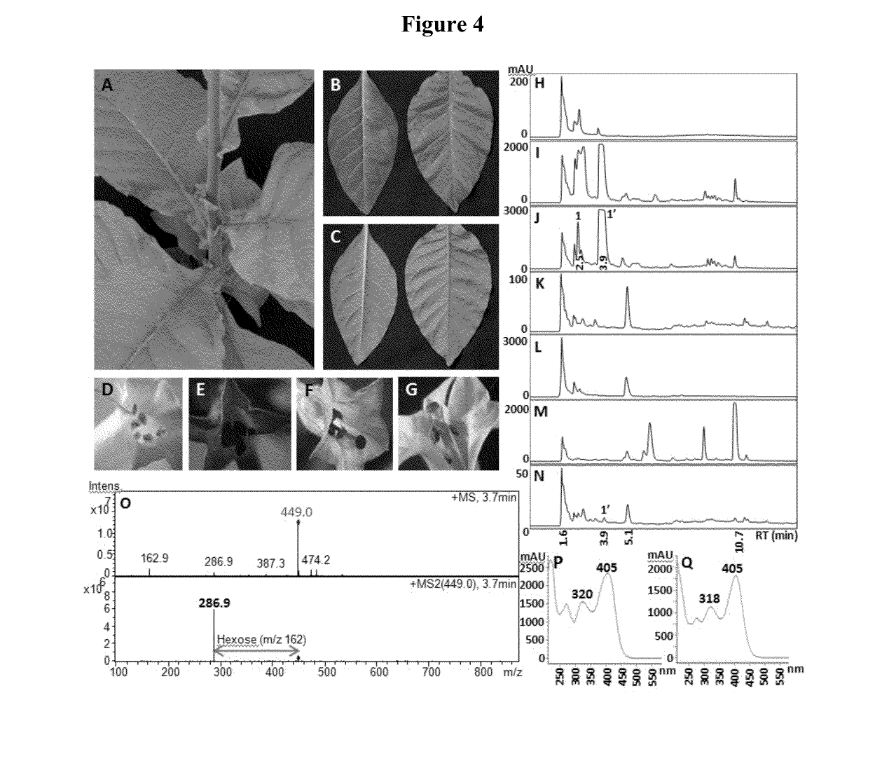

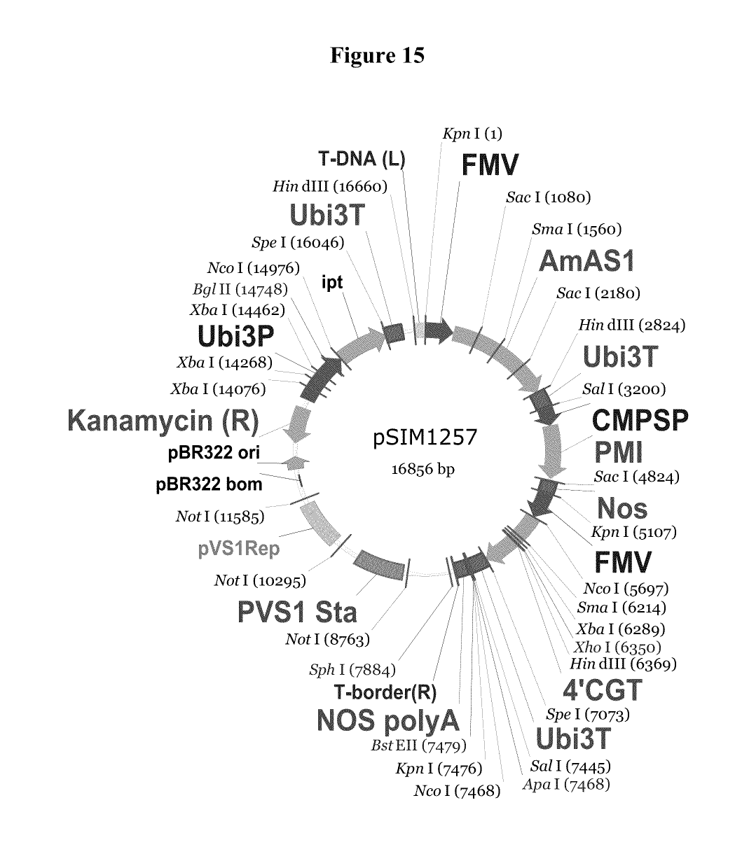

The naringenin chalcone-rich plants were transformed a third time with the T-DNA of pSIM1257 (FIG. 15), which carry the aurone biosynthetic genes (similar to pSIM1251, except that pmi was replaced with the hygromycin phosphotransferase selectable marker gene, hpt). Transformed cells proliferated only on tissue culture media supplemented with naringenin chalcone and developed bright yellow calli, suggesting an effective conversion of the plant-produced compound to AOG. Subsequent regeneration produced yellow-green shoots that were markedly different from the green-purple shoots of parental lines. Upon planting in soil, these shoots started to accumulate some purple pigments, indicative a lingering Chi activity, so that leaves of triply-transformed plants appeared bronze-green (FIG. 4A-C). Unlike, the pink or purple flowers of control and parental lines (FIG. 4D-F), and these plants produced yellow-orange colored flowers (FIG. 4G). The bronze-green leaves of 646/1252/1257 lacked detectable amounts of the anthocyanins and flavanones that were abundant in 646/1252 lines expressing the StMtf1^{M} gene and partially silenced for Chi (FIG. 4H-I). Confirming our earlier assumption, these compounds were converted into AOG. The yellow aurone compound had accumulated in leaves to levels nearly two-thirds those in snapdragon flowers (FIG. 4J, peak 1 and 1′, and Table 1). The parental 646/1252 line lacked these peaks (FIG. 4K-M). Interestingly, over-expressing the Am4CGT and AmAs1 genes in deep purple plants (646/1257) without silencing Chi produced only trace amounts of AOG (FIG. 4N). The UV-diode array detection (UV-DAD) profile of AOG of 646/1252/1257 (FIG. 4O and P) and MS/MS fragmentation (FIG. 4Q) of peak 1′ at a retention time of 3.9 min were identical to both snapdragon AOG and commercially-available maritimein (3′,4′,6,7-tetrahydroxy-6-O-glycosylaurone or maritimetin-6-O-glucoside). The trace amount of compound with molecular ion at m/z 465 was co-eluting with AOG, peak 1′ in both 646/1252/1257 leaves and snapdragon flower which has the same UV maximum as that of AOG tentatively and identified as bracteatin-6-glucose.

### Example 4

### Aureusidin Formation in Lettuce Plants Expressing the Am4′CGT and AmAs1 Genes

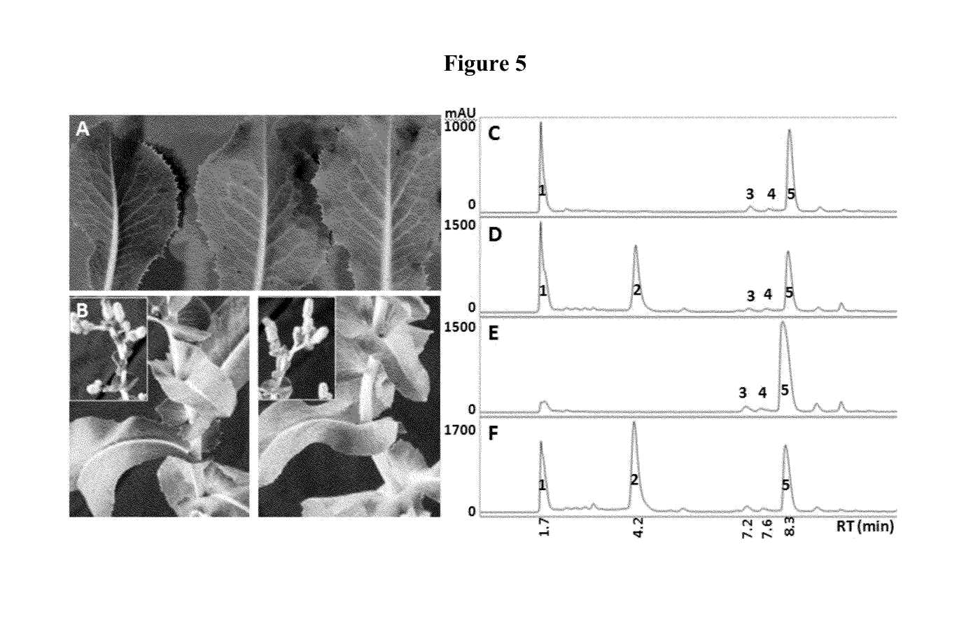

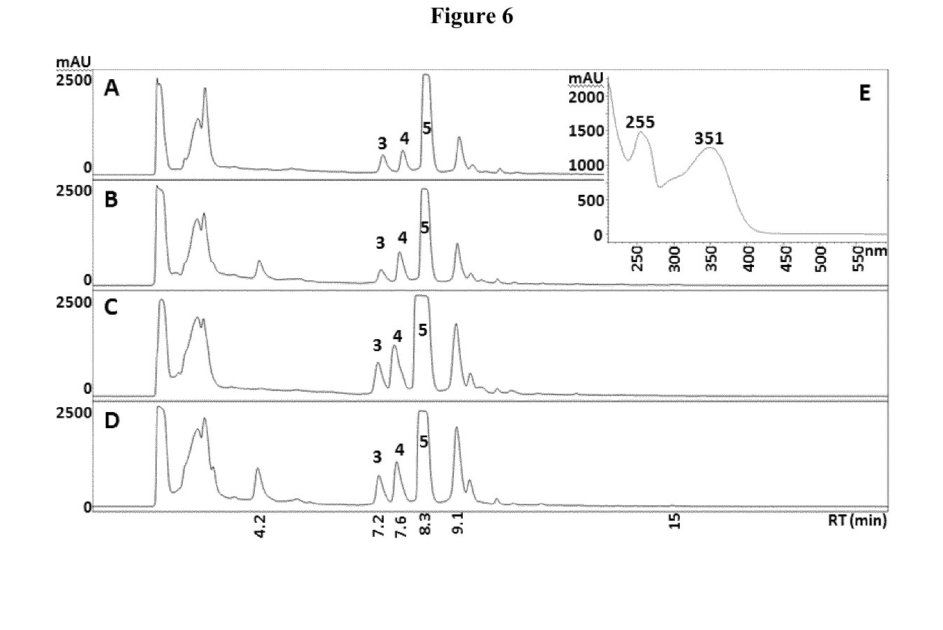

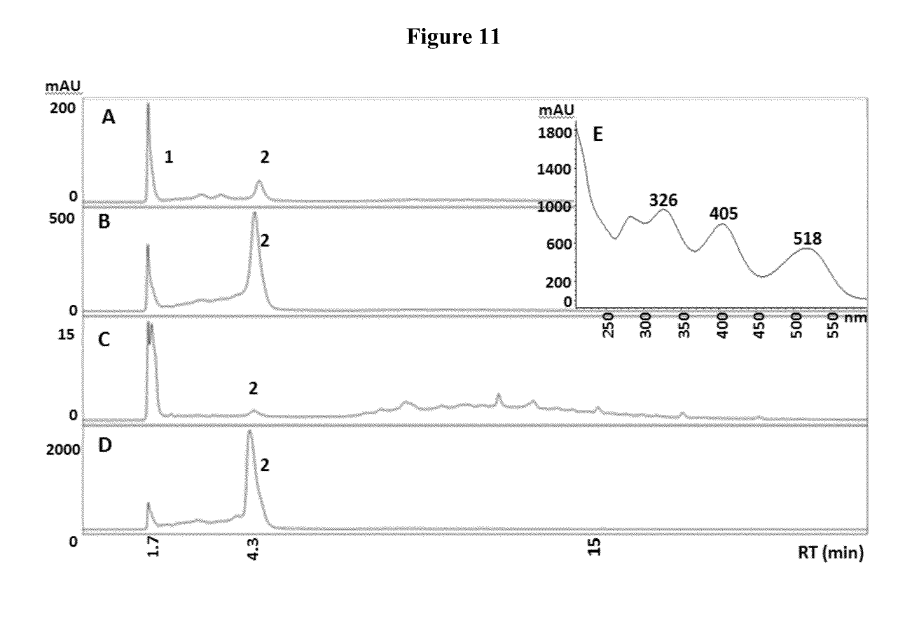

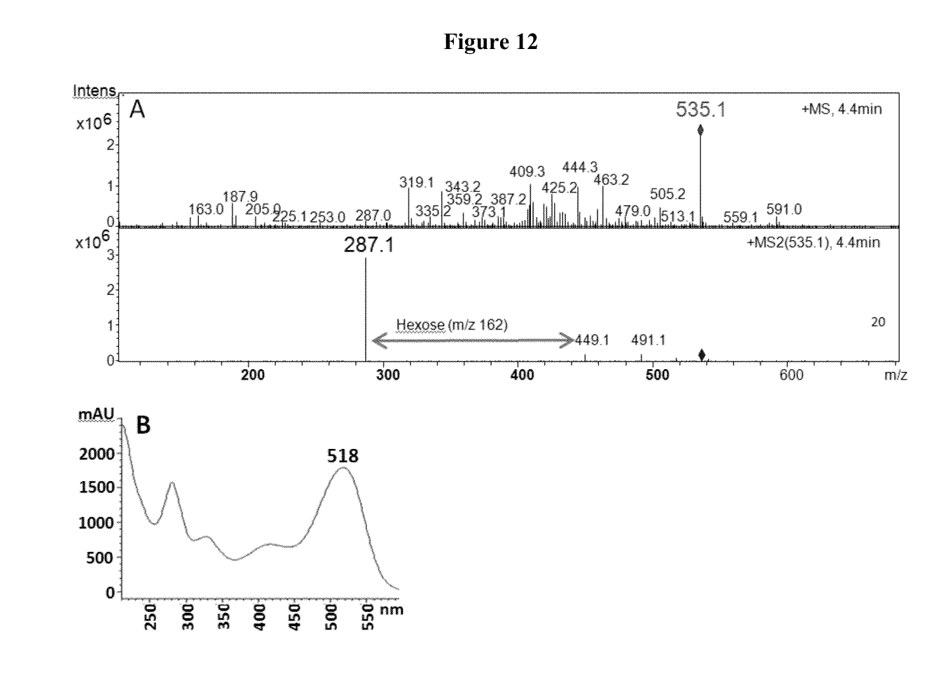

The lettuce Lactuca sativa cultivar “Eruption” produces purple leaves. Plants of this variety were transformed to express the Am4′CGT and AmAs1 genes (pSIM1610). Upon transfer to the greenhouse, leaf color turned bronze-green (FIG. 5A-B). The leaves of these transgenic plants (1610) were demonstrated by LC/MS to contain a large amount of AOG, which is not present in the untransformed control (FIG. 5C-D, peak 2). The associated peak co-eluted with an anthocyanin compound that also accumulated in untransformed plants, as shown in the HPLC chromatogram in FIG. 11A-D and the UV spectrum in FIG. 11E. LC/MS-MS detection in positive ionization modes was used to obtain more information on compound structure. The co-eluted compound in peak 2 was attributed to cyanidin-3-(6′-malonyl) glucoside, based on MS/MS fragmentation (m/z 535, MS² fragments, 449 and 287 corresponding to loss of first 86 amu, i.e., the malonyl moiety, and then 162 amu, i.e., the hexose moiety) (FIG. 12A) and the UV spectrum (FIG. 12B). Aureusidin-6-O-glucoside and cyanidin-3-(6′-malonyl)-glucoside were quantified as shown in Table 2. Silencing of the Dfr (dihydro flavonol 4-reductase) gene (pSIM1618) in wild-type lettuce almost completely blocked the formation of this anthocyanin. As expected, retransformation of the Dfr-silenced plants (1618) with the Am4′CGT and AmAs1 genes (pSIM1610) resulted in AOG formation. The amount of AOG was slightly higher in 1610/1618 lettuce plants than in plants that were not silenced for Dfr (FIG. 5E-F). LC/MS and MS-MS data tentatively identified three main flavonoids (denoted as peak 3, 4 and 5) in wild-type lettuce as a quercetin derivative (m/z 479, product ion 303), kaempferol-glucoside (m/z 463, product ions 463 & 287) and quercetin-3-(6′-malonyl) glucoside (m/z 551, product ions 465 & 303) respectively. The amount of flavonoids did not change significantly upon overexpression of Am4′CGT and AmAs1, regardless of whether or not Dfr was silenced. The HPLC chromatograms are illustrated in FIG. 6 A-D and the UV spectrum showed the absorption maximum of major flavonoid peak 5 at 255 and 351 nm (FIG. 6E).

### Example 5

### Aureusidin Formation is Linked to Enhanced Dismutase Activity

The peroxyl radical scavenging capacity of transgenic control plants was 12 mole equivalents of the vitamin E analog Trolox (TE) gram⁻¹. This value is similar to those of most vegetables (Song et al., 2010). As shown in Table 3, activation of the anthocyanin biosynthetic pathway in ANT1 plants resulted in a 2.5-fold increase in ORAC value (to 29 moles TE gram⁻¹), to levels that are typical for common fruits, such as orange and grape (Wolfe et al., 2008). Interestingly, the almost complete conversion of anthocyanins to aurones that was accomplished in 646/1252/1257 plants resulted in a much greater increase in ORAC values, to an average of 78 mmoles TE gram⁻¹. These levels resembled those of various berries, such as blueberry, blackberry and raspberry that provide the highest known antioxidant activities of any edible food (Wu et al., 2004; Wolfe et al., 2008).

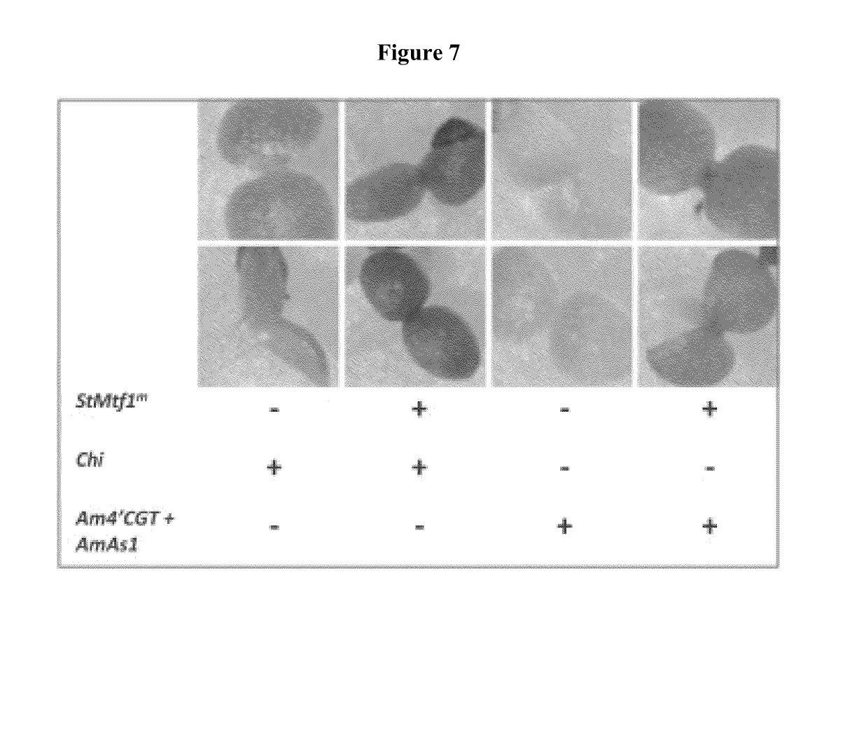

Self-fertilization of the triply transformed TO plants produced segregating T1 families with various seedling colors (FIG. 7). Seedlings with a bronze-green color, confirmed to contain at least one copy of each of the three constructs used for transformation, were allowed to develop into mature plants in the greenhouse. ORAC analysis confirmed unusually high antioxidant activities of, on average, 54.2 M TE gram⁻¹ in leaves of randomly selected T1 plants. Similar results were obtained for homozygous T2 plants (Table 3).

Because superoxide free radicals are at least as important in triggering oxidative stress as peroxyl radicals, we employed a xanthine-xanthine oxidase system with a tetrazolium salt as reducing agent to assess the capacity of plants to scavenge such O₂⁻ anions. As shown in Table 4, leaf extracts of transgenic T0 and T1 control plants inhibited SOD by 27% and 25.5%, respectively. This inhibitory activity increased slightly, to 36%, when extracts of the anthocyanin-rich T1 leaves of StMtf1^{M} plants (ANT1) were used, whereas no increase in inhibitory activity was found in T0 leaves. However, the conversion of most of the anthocyanins to aurones resulted in superior SOD inhibiting activities of up to 90% in T0 and 50-60% in T1 plants of two 646/1252/1257 lines (Table 4). Homozygous AOG-producing T2 plants continued to display high SOD inhibiting activities (62-77%) compared to their transgenic controls (24%).

Antioxidant activities were also determined in aurone-overexpressing lettuce in the presence and absence of the Dfr gene. As shown in Table 5, SOD inhibition was three-fold greater in T0 aurone-expressing lettuce (1610/1618) than in wild-type and transgenic lettuce controls (1610 and 1618). All T1 lettuce plants that overexpressed aurone (1610), were silenced for Dfr (1618) and both overexpressed aurone and were silenced for Dfr (1610/1618) showed a two-fold inhibition of SOD inhibition compared to controls. Similar results were obtained with the ORAC assay performed on T1 transgenic lettuce leaves.

We demonstrated that the aurone biosynthetic pathway can be transferred from flowers of the ornamental plant snapdragon to the vegetative tissues of tobacco and lettuce. In addition to the expression of the snapdragon Am4′CGT and AmAs1 genes, aurone formation in tobacco required modifications, that increased the accumulation of the flavonoid naringenin chalcone which is the substrate for Am4′CGT. These modifications involved increasing StMtf1^{M} gene expression and lowering the expression of the Chi gene. Although transformed cells produced large amounts of aurones in tissue culture, developing bright yellow calli, it was difficult to subsequently regenerate transgenic shoots. Indeed, aurone-producing tobacco plants were obtained only when tissue culture media were supplemented with naringenin chalcone. These results confirm the important role that flavonoids play in mediating auxin transport (Peer and Murphy, 2007). Chi gene silencing was unnecessary in the lettuce variety “Eruption”, which has a functionally active flavonoid biosynthetic pathway and naturally produces anthocyanins. However, aurone formation was effectively enhanced upon silencing of the alternative gene, Dfr. The presence of cyanidin-3-(6′-malonyl) glucoside in the doubly transformed 1610/1618 lines was due to the partial silencing of Dfr. These data were supported by the ammonia test (Lawrence, 1929), which detects anthocyanins in plant tissues (data not shown).

Our data demonstrated that the ability of crops to produce aurone broadens their diversity of dietary antioxidants and increases their nutritional value. We evaluated for the first time the antioxidant activity of aurone in lettuce and tobacco plants. Food crops produce antioxidants and the dietary intake of these antioxidants is important for health. Currently available crop varieties have not been optimized for their total antioxidant power, and efforts to increase this important trait through genetic modification are generally limited to less than a two-fold increase (Reddy et al., 2007; Aksamit-Stachurska e al., 2008). Although aurones are simple flavonoid compounds, their biosynthesis is associated with a significant increase in total antioxidant power. Indeed, the novel strategy presented in this study increased the total antioxidant power by up to seven-fold.

Under stress conditions, aurone-containing plants have even higher free radical scavenging activity, because stress induced flavonoid biosynthesis in plants (Ebel, 1986; Shirly 2002). Our data support the notion that aureusidin-6-O-glucose formation is enhanced under conditions of nutrient limitation. All controls, aurone-overexpressing lines and Dfr-silenced lines were stunted in growth, displayed accelerated flowering, and produced lower amounts of purple pigments during nutrient limitation than when normal amounts of fertilizer were applied. These changes had a negative effect on the antioxidant activities of transgenic controls and aurone lines (data not shown). However, the imposed abiotic stress was correlated with an increased formation of yellow pigment in double transformants. These plants displayed an increased capacity to scavenge peroxyl radicals and inhibit SOD.

We demonstrated in this study that aurone formation not only increases the diversity of antioxidants present in a plant, but also likely represents a beneficial consumer trait. The fruits and vegetables that are most frequently consumed in the United States, such as apples and potatoes, are known to be poor sources of phytonutrients (DeWeerdt, 2011). There is an inverse association between the total intake of fruits and vegetables and the risk of developing cancer (Boffetta et al., 2010) and coronary heart disease (Dauchet et al., 2006). This health-promoting effect has been attributed to the additive and/or synergistic activity of mixtures of antioxidants (Liu, 2004; Messina et al., 2001; http://www.cnpp.usda.gov/dietaryguidelines.htm). Our data suggest that aurones can be produced in any fruit or vegetable crop that produces at least some naringenin chalcone. This could be new and natural source of fruits and vegetables mainly due to the numerous additive and synergistic effects of such compounds. Until now, aurones have been considered only as a means to enhance the color of ornamental flowers. Transferring the capacity to produce specific antioxidants across plant species through genetic engineering could compensate for the lack of diversity in many modern diets. This study presents a strategy for developing a novel class of functional foods.

### REFERENCES

Aksamit-Stachurska A., Korobczak-Sosna A., Kulma A. and Szopa J. (2008) Glycosyltransferase efficiency controls phenylpropanoid pathway. BMC Biotechnol. 8, 25.
Aswath C. R., Mo S. Y., Kim D. H. and Park S. W. (2005) Agrobacterium and biolistic transformation of onion using non-antibiotic selection marker phosphomannose isomerase. Plant Cell Rep. 25, 92-9.
Bjelakovic G. and Gluud C. (2007) Surviving antioxidant supplements. J. Natl. Cancer Inst. 99, 742-743.
Boffetta P., Couto E., Wichmann J., Ferrari P., Trichopoulos D., Bueno-de-Mesquita H. B. and van Duijnhoven F. J. (2010) Fruit and vegetable intake and overall cancer risk in the European Prospective Investigation into Cancer and Nutrition (EPIC). J. Natl. Cancer Inst. 102, 529-537.
Boumendjel A. (2003) Aurones: a subclass of flavones with promising biological potential. Curr. Med. Chem. 10, 2621-2630.
Dauchet L., Amouyel P., Hercberg S, and Dallongeville J. (2006) Fruit and vegetable consumption and risk of coronary heart disease: a meta-analysis of cohort studies. J. Nutr. 136, 2588-2593.
Detsi A., Majdalani M., Kontogiorgis C., Hadjipavlou-Litina D. and Kefalas P. (2009) Natural and synthetic 2′-hydroxy-chalcones and aurones:synthesis, characterization and evaluation of antioxidant and soybean lipoxygenase inhibitor activity. Bioorg. Med. Chem. 17, 8073-8085.
DeWeerdt S. (2011) Food: The omnivore's labyrinth. Nature 471, S22-24.
Ebel J. (1986) Phytoalexin synthesis: The biochemical analysis of the induction process. Annu. Rev. Phytopathol. 24, 235-264.
Ferreira E. O., Salvador M. J., Pral E. M., Alfieri S. C., Ito I. Y., Dias D. A. (2004) A new heptasubstituted (E)-aurone glucoside and other aromatic compounds of Gomphrena agrestis with biological activity. Z naturforsch C. 59, 499-505.
General books LLC. (2010) Amaranthaceae: Sugar beet, spinach, salsoda soda, halothamnus barilla, halothamus subaphyllus, tecticomia, halothamnus glaucus, maireana. New York: General books LLC.
Harborne J. B. and Smith D. M. (1978) Anthochlors and other flavonoids as honey guides in the Compositae. Biochem. Syst. Ecol. 6, 287-291.
Haudecoeur R. and Boumendjel A. (2012) Recent advances in the medicinal chemistry of aurones. Curr Med Chem 19, 2861-2875.
Haung D., Ou B. and Prior R. L. (2005) The chemistry behind antioxidant capacity assays. J. Agric. Chem. 53, 1841-1865.
Iwashina T. (2000) The structure and distribution of the flavonoids in plants. J. Plant Res. 113, 287-299.
Lawrence, W. J. C. (1926) The genetics and cytology of Dahlia species. J. Genet. 21, 125-159.
Liu R. H. (2004) Potential synergy of phytochemicals in cancer prevention: mechanism of action. J. Nutr. 134, 3479S-3485S.
Lunau K., Wacht S, and Chittka L. (1996) Colour choices of naive bumble bees and their implications for colour perception. J. Comp. Physiol. A 178, 477-489.
Messina M., Lampe J. W., Birt D. F., Appel L. J., Pivonka E., Berry B. and Jacobs D. R. Jr (2001) Reductionism and the narrowing nutrition perspective: time for reevaluation and emphasis on food synergy. J. Am. Diet. Assoc. 101, 1416-1419.
Milovanovic M., Picuric-Jovanovic K., Djermanovic V. and Stefanovic M. (2002) Antioxidant activities of the constinuents of Picris echoides. J. Serb. Chem. Soc. 66, 7-17.
Nakayama T., Yonekura-Sakakibara K., Sato T., Kikuchi S., Fukui Y., Fukuchi-Mizutani M., Ueda T., Nakao M., Tanaka Y., Kusumi T., Nishino T. (2000) Aureusidin synthase: a polyphenol oxidase homolog responsible for flower coloration. Science 290 (5494):1163-6.
Ono E., Hatayama M., (sono Y., Sato T., Watanabe R., Yonekura-Sakakibara K., Fukuchi-Mizutani M., Yanaka Y., Kusumi T. and Nishino T. (2006) Localization of a flavonoid biosynthesis polyphenol oxidase in vacuoles. Plant J. 45, 133-143.
Peer W. A. and Murphy A. S. (2007) Flavonoids and auxin transport: modulators or regulators? Trends Plant Sci., 12, 556-63.
Prior R. L., Hoang H., Gu L., Wu X., Bacchiocca M., Howard L., Hampsch-Woodill M., Huang D., Ou B., and Jacob R. (2003) Assays for hydrophilic and lipophilic antioxidant capacity (oxygen radical absorbance capacity (ORACFL) of plasma and other biological and food samples. J. Agric. Food Chem. 51, 3273-3279.
Rathmell W. G. and Bendall D. S. (1972) The peroxidase-catalyzed oxidation of a chalcone and its possible physiological significance. Biochem. J. 127, 125-132.
Reddy A. R., Reddy A. M., Reddy V., Scheffler B., Wienand U. (2007) Novel transgenic rice overexpressing anthocyanin synthase accumulates a mixture of flavonoids leading to an increased antioxidant potential. Metabolic Engineering 9, 95-111.
Richael C. M., Kalyaeva M., Chretein R. C., Yan H., Adimulam S., Stivision A., Weeks J. T. and Rommens C. M. (2008) Cytokinin vectors mediate marker-free and backbone-free plant transformation. Transgenics Res. 17, 905-917.
Rodriguez-Saona L. E., and Wrolstad R. E. (2001) Extraction, Isolation, and purification of anthocyanins. Current Protocols in Food Analytical Chemistry unit F1.1.1-11.
Rommens C. M., Richael C. M., Yan H., Navarre D. A., Ye J., Krucker M. and Sword K. (2008) Engineered native pathways for high kaempferol and caffeoylquinate production in potato. Plant Biotech J., 6, 870-886.
Saito N., and Ine S. S. (1990) Breeding of carnation on flower color pigment and green flower color. Biohorti 1, 49-57.
Shirley W. B. (2002) Biosynthesis of flavonoids and effects of stress. Curr. Opin. Plant Biol. 5, 218-23.
Song W., Derito C. M., Liu M. K., He X., Dong M. and Liu R. H. (2010) Cellular antioxidant activity of common vegetables. J. Agric. Food Chem. 58, 6621-6629.
Venkateswarlu S., Panchagnula G. K. and Subbaraju G. V. (2004) Synthesis and antioxidative activity of 3′,4′,6,7-tetrahydroxyaurone, a metabolite of Bidens frondosa. Biosci Biotechnol Biochem 68, 2183-2185.
Whibley A. C., Langlade N. B., Andalo C., Hanna A. I., Bangham A., Thébaud C. and Coen E. (2006) Evolutionary paths underlying flower color variation in Antirrhinum. Science 313, 963-966.
Wolfe K. L., Kang X., He X., Dong M., Zhang Q. and Liu R. H. (2008) Cellular antioxidant activity of common fruits. J. Agric. Food Chem. 56, 8418-8426.
Wong E. (1966) Occurrence and biosynthesis of 4′,6-dihydroxyaurone in soyabean. Phytochem. 5, 463-467.
Wu X., Beecher G. R., Holden J. M., Haytowitz D. B., Gebhardt S. E. and Prior R. L. (2004) Lipophilic and hydrophilic antioxidant capacities of common foods in the United States. J. Agric. Food Chem. 56, 4026-4037.
Zwergel C., Gaasscht F., Valente S., Diederich M., Bagrel D., kirsch G. (2012) Aurones: interesting natural and synthetic compounds with amerging biological potential. Nat prod Commun 7.389-394.

### TABLES

TABLE 1
Analyses of flavonoids and anthocyanins in aurone extracts of transgenic tobacco leaves.

| | | HPLC chromatogram at different DAD wavelengths | | | | | | | | | |
|---|---|---|---|---|---|---|---|---|---|---|---|
| | | Aurone | | Anthocyanin | | | | | | | |
| | | at 400 nm | | at 520 nm | | Flavonoids at 360 nm | | | | | |
| | | Peak 1 | Peak 1′ | Peak 2 | Peak 3 | Peak 4 | Peak 5 | Peak 6 | Peak 7 | Peak 8 | |
| | | 2.6 min | 3.9 min | 2.6 min | 2.6 min | 5.1 min | 5.9 min | 6.2 min | 9.1 min | 9.7 min | Peak 9 |
| Transgenic | Ind. | AOG | AOG | NID | Cyn-ru | PHF-glu | NC deriv | NC-diglu | NC-glu | TMC-glu | NC |
| lines and | line | mg/g | mg/g | mg/g | mg/g | mg/g | mg/g | mg/g | mg/g | mg/g | mg/g |
| Controls | # | DW | DW | DW | DW | DW | DW | DW | DW | DW | DW |

Leaves

| | | | | | | | | | | | |
|---|---|---|---|---|---|---|---|---|---|---|---|
| Wild type | 1 | — | — | — | — | 1.6 | — | — | 0.005 | — | 0.0006 |
| 646 | 1 | — | — | 0.076 | 0.137 | 19.89 | — | — | 0.079 | — | 0.0005 |
| 646/1252 | 1 | — | — | 0.002 | 0.004 | 10.1 | 7.8 | 4.3 | 4.5 | trace | 2.3 |
| | 3 | — | — | 0.006 | 0.006 | 4.01 | 42.8 | 7.8 | 9.0 | trace | 2.5 |
| | 5 | — | — | 0.003 | 0.003 | 5.52 | 10.2 | 6.1 | 4.9 | trace | 2.3 |
| 646/1251 | 2 | — | trace | — | — | 0.55 | — | — | 0.006 | trace | 0.0011 |
| | 9 | — | trace | — | — | 1.49 | | — | 0.005 | trace | 0.0015 |
| | 11 | — | trace | — | — | 0.89 | | — | 0.002 | trace | 0.006 |
| 646/1252/1257 | 14 | 0.268 | 2.04 | 0.006 | 0.01 | 1.6 | — | 0.03 | 1.2 | 0.52 | 0.43 |
| | 19 | 0.039 | 2.05 | 0.003 | 0.05 | 3.0 | | 0.01 | 1.4 | 0.58 | 0.8 |
| | 27 | 0.018 | 1.89 | 0.003 | 0.03 | 2.6 | | 0.05 | 1.0 | 0.56 | 0.1 |

Flowers

| | | | | | | | | | | | |
|---|---|---|---|---|---|---|---|---|---|---|---|
| 646/1252/1257 | 1 | — | 0.13 | — | 0.003 | 0.95 | — | 14.08 | 7.8 | — | 1.5 |
| Snapdragon | 1 | 0.649 | 3.45 | — | — | — | — | — | — | — | |

Tentative peak identification: Peaks 1 and 1′, aureusidin-6-O-glucose (AOG); Peak 2, unidentified anthocyanin (NID); Peak 3, cyanidin-rutinose (Cyn-ru); Peak 4, pentahydroxy flavone glucose (PHF-glu); Peak 5, naringenin chalcone (NC) derivative, Peak 6, naringenin chalcone diglucoside (NC-diglu); Peak 7, naringenin chalcone glucose (NC-glu); Peak 8, tetrahydroxy methoxy chalcone glucose (TMC-glu); Peak 9, naringenin chalcone (NC).

TABLE 2
Analyses of flavonoids and anthocyanins in aurone extracts of transgenic lettuce leaves.

| | | HPLC chromatogram at different DAD wavelengths | | |
|---|---|---|---|---|
| | | Peak 2 @400 nm | Peak 2@520 nm | Peak 5@360 nm |
| | | 4.3 min | 4.4 min | 8.3 min |
| | | Aureusidin-6-O- | Cyanidin-3-(6′- | Quercetin-3-(6′- |
| Transgenic lines and | Independent | glucoside | malonyl) glucoside | malonyl) glucoside |
| Controls | lines # | mg/g DW | mg/g DW | mg/g DW |

Leaves

| | | | | |
|---|---|---|---|---|
| Wild type | 1 | 0.000 | 0.012 | 6.073 |
| Transgenic control | A | 0.000 | 0.014 | 22.609 |
| | B | 0.000 | 0.218 | 22.599 |
| 1610 | K | 0.173 | 0.299 | 17.857 |
| | M | 0.311 | 0.296 | 19.889 |
| | O | 0.076 | 0.151 | 16.020 |
| 1618 | V | 0.000 | 0.000 | 22.203 |
| | K | 0.000 | 0.070 | 22.968 |
| 1610/1618 | 2A | 0.710 | 0.762 | 23.878 |
| | 2K | 0.192 | 0.205 | 17.047 |
| | 3C | 0.296 | 0.004 | 27.974 |

Flowers

| | | | | |
|---|---|---|---|---|
| Snapdragon petals | 1 | — | 3.76 | — |

— indicates not detected.

TABLE 3
Gene type and oxygen radical absorbance capacity (ORAC)
of transgenic tobacco leaves and their control

| | Genes | | | | ORAC assay (μmoles TE/g) | | |
|---|---|---|---|---|---|---|---|
| Line | StMtf1M | Chi | Am4CGT | AmAs1 | T0 | T1 | T2 |
| Transgenic control | + | + | − | − | 12.2 | 20.8 | 13.6 |
| 646 | + | + | − | − | 28.8 | 19.2 | 95 |
| 646/1252 | + | +/− | − | − | | | 113.3 |
| 646/1252/1257-12, 14, 19 | + | +/− | + | + | 78 | 54.2 ± 5.0 | 83.5 ± 9 |
| 646/1252/1257-26, 27, 31 | + | +/− | + | + | | | 103.3 ± 57 |

ORAC data expressed as micromoles of Trolox equivalent per gram (μmoles of TE/g).
+ and − indicates for presence and absence of corresponding gene.
+/− indicates for presence or absence of Chi gene varies in different independent lines.

TABLE 4
Gene type and superoxide radical scavenging capacity (SOD
inhibition) of transgenic tobacco leaves and their control.

| | Genes | | | | SOD activity (inhibition rate %) | | |
|---|---|---|---|---|---|---|---|
| Line | StMtf1M | Chi | Am4CGT | AmAs1 | T0 | T1 | T2 |
| Transgenic Control | + | + | − | − | 27 | 25.5 ± 0.5 | 29 |
| 646 | + | + | − | − | 19 | 36 ± 5.0 | 55 |
| 646/1252 | + | +/− | − | − | | | 60 |
| 646/1252/1257 -12, 14, 19 | + | +/− | + | + | 91 | 60.3 ± 5.9 | 69 |
| 646/1252/1257-26, 27, 31 | + | +/− | + | + | | 50.0 ± 6.1 | 62 |

TABLE 5
Gene type, oxygen radical absorbance capacity (ORAC)
and superoxide radical scavenging capacity (SOD inhibition)
of transgenic tobacco leaves and their control.

| | | | | SOD activity | | ORAC assay |
|---|---|---|---|---|---|---|
| | Genes | | | (inhibition rate %) | | (μmoles TE/g) |
| Line | Dfr | Am4CGT | AmAs1 | T0 | T1 | T1 |
| Wild type | − | − | − | 28.5 | 34 | |
| Transgenic control | − | − | − | 31.7 | 35 | 14.068 |
| 1610 | | + | + | 80.5 | 56 | 11.54 |
| 1618 | + | − | − | | | 15.2 |
| 1610/1618 | + | + | + | 90 | 59 | 22.2 |

ORAC data expressed as micromoles of Trolox equivalent per gram (μmoles of TE/g).
+ and − indicated for presence and absence of the corresponding gene.

What is claimed is:

1. A method for modifying a plant, comprising overexpressing or expressing de novo at least one of (i) chalcone 4′-O-glucosyltransferase, and (ii) aureusidin synthase, in said plant.

2. The method of claim 1, comprising overexpressing or expressing de novo potato both chalcone 4′-O-glucosyltransferase and aureusidin synthase in said plant.

3. The method of claim 1, wherein the chalcone 4′-O-glucosyltransferase and/or aureusidin synthase is expressed in the flowers of said plant.

4. The method of claim 1, wherein the chalcone 4′-O-glucosyltransferase and/or aureusidin synthase is expressed in the leaves of said plant.

5. The method of claim 1, wherein the chalcone 4′-O-glucosyltransferase is Antirrhinum majus chalcone 4′-O-glucosyltransferase, and wherein the aureusidin synthase is Antirrhinum majus aureusidin synthase.

6. The method of claim 1, comprising (A) stably integrating into the genome of at least one plant cell (a) an exogenous gene expression cassette for expressing chalcone 4′-O-glucosyltransferase and (b) an exogenous gene expression cassette for expressing aureusidin synthase, and (B) regenerating the transformed plant cell into a plant.

7. The method of claim 1, further comprising overexpressing or expressing de novo one or more genes involved in the biosynthesis of naringenin chalcone to increase the production of naringenin chalcone in said plant.

8. The method of claim 1, further comprising downregulating one or more genes involved in the conversion of naringenin chalcone to anthocyanin to decrease the consumption of naringenin chalcone for anthocyanin biosynthesis.

9. The method of claim 1, comprising (A) overexpressing or expressing de novo chalcone 4′-O-glucosyltransferase and aureusidin synthase, (B) overexpressing or expressing de novo potato transcription factor StMtf1^{M}, and optionally (C) downregulating the expression of chalcone isomerase and/or dihydro flavonol 4-reductase.

10. The method of claim 1, wherein the leaves of the modified plant produce at least 100% more aureusidin-6-O-glucoside than the leaves of a wild plant of the same variety.

11. A modified plant comprising in its genome one or more exogenous genetic cassettes selected from the group consisting of (i) a gene expression cassette for expressing chalcone 4′-O-glucosyltransferase, and (ii) a gene expression cassette for expressing aureusidin synthase.

12. The plant of claim 11, comprising in its genome both (i) the gene expression cassette for expressing chalcone 4′-O-glucosyltransferase, and (ii) the gene expression cassette for expressing aureusidin synthase.

13. The plant of claim 11, further comprising in its genome (iii) an exogenous gene expression cassette for expressing at least one gene involved in the biosynthesis of naringenin chalcone; and/or (iv) an exogenous gene silencing cassette for downregulating at least one gene involved in the conversion of naringenin chalcone to anthocyanin.

14. The plant of claim 11, wherein the plant is a leaf vegetable.

15. The plant of claim 11, wherein (a) the leaves of the plant produces at least 100% more aureusidin-6-O-glucoside than the leaves of a wild plant of the same variety, and (b) the aureusidin-6-O-glucoside concentration in the leaves of the plant is at least 10% of the aureusidin-6-O-glucoside concentration in the flowers of a wild plant of Antirrhinum majus.

16. The plant of claim 11, wherein the leaves of the plant have (a) at least 50% higher super oxide dismutase (SOD) inhibiting activities, and (b) at least 50% higher oxygen radical absorbance capacity (ORAC) activities, compared to the leaves of a wild plant of the same variety.

17. A food product or nutritional composition produced from the plant of claim 15.

18. A transformation vector comprising one or more genetic cassettes selected from the group consisting of (i) a gene expression cassette for expressing chalcone 4′-O-glucosyltransferase, and (ii) a gene expression cassette for expressing aureusidin synthase.

19. The transformation vector of claim 18, comprising a first gene expression cassette for expressing Antirrhinum majus chalcone 4′-O-glucosyltransferase, and a second gene expression cassette for expressing Antirrhinum majus aureusidin synthase.

20. A method comprising: (A) stably integrating into the genome of at least one plant cell (i) an exogenous gene expression cassette for expressing chalcone 4′-O-glucosyltransferase and (ii) an exogenous gene expression cassette for expressing aureusidin synthase, and (B) proliferating the transformed plant cell in the presence of naringenin chalcone.
